SEQUENCE LISTING (28 sequences)

SEQ ID NO: 1 - DNA, 408 bp, Mus musculus
tcttgcccatcggggccacggatacctgtgtgtcctccccgccattccatgcccaacggggttttggatacttacctgccttttcattctttttttttcttattatttttttttctaaacttgcccatctgggctgtggatacctgcttttattctttttttcttctccttagcccatcggggccatggatacctgctttttgtaaaaaaaaaaaaaaaaacaaaaaaacctttctcggtccatcgggacctcggatacctgcgtttagtctttttttcccatgcccaacggggcctcggatacctgctgttattatttttttttctttttcttttgcccatcggggctgtggatacctgctttaaattttttttttcacggcccaacggggcgcttggtggatggaa

SEQ ID NO: 2 - DNA, 395 bp, R. norvegicus
ttttgcccatcggggccacggatacctgtgtgtcctcccagccattccatgtccagctgggcttgggatacttaacctgccttttaatccttttttcttcttcttacttttcttcttctaaacttgcccatctgggttgtggatacctgcttttattctttttttcttcttctccttagcccatcggggccatggatacctgctttttaccaaaaaacgccgtatttctcggtccatcgggacctcggatacctgcgtttagtttttttcccatgcccaacggggcctcggatacctgctttaatttttttttcttttccttttgcccatcggggctgtggatacctgctttaattttttttttcacggcccatcggggcatttggtggatggaa

SEQ ID NO: 3 - DNA, 434 bp, Felis catus
ttttgcccatcggggctgtggataccaggttttattatcatttttttcgcccaacggggctgtggatacctgcgttttaattcttttcttttatttattttttttaatttgcccatcggggcagcggatacctgcttttaattttttttttttttcacccttagcccatcggggcctcggatacctgctgtgtctccttccctccccttaaccctctggcccatcggggcaatggataccagctttaaaaaaagttcctttttggcccatcggggcctcggatacctgcttttattattttttttcccttgcccatcggggccttggatacctgctttatttatttattttttttcctttgcccatcggggctgtggatacctgcttagatttttttttttctcatggcccatcggggccttttatggatggaa

SEQ ID NO: 4 - DNA, 408 bp, Homo sapiens
ttttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaa

SEQ ID NO: 5 - RNA, 15 nt, Artificial sequence
  Synthetic oligonucleotide
  misc_feature (1)..(15): y is c or t
  misc_feature (1)..(15): n is any base
nngyccaucggggyn

SEQ ID NO: 6 - RNA, 13 nt, Artificial sequence
  Synthetic oligonucleotide
  misc_feature (1)..(13): n is any base
  misc_feature (1)..(13): y is c or t
nyggauaccugnn

SEQ ID NO: 7 - protein, 12 aa, Mus musculus
  MISC_FEATURE (1)..(12): X is any amino acid
LLXXGDVENPGP

SEQ ID NO: 8 - protein, 12 aa, Mus musculus
  MISC_FEATURE (1)..(12): X is any amino acid
LLXXGDVSNPGP

SEQ ID NO: 9 - protein, 13 aa, Mus musculus
  MISC_FEATURE (1)..(13): X is any amino acid
LLXXXGDVENPGP

SEQ ID NO: 10 - protein, 12 aa, Mus musculus
  MISC_FEATURE (1)..(12): X is any amino acid
LLXXGDVSNPGP

SEQ ID NO: 11 - DNA, 20 bp, Artificial sequence
  Synthetic oligonucleotide
gctcgtttcccgtggatgtg

SEQ ID NO: 12 - DNA, 20 bp, Artificial sequence
  Synthetic oligonucleotide
gtggatgtgcggttcttccg

SEQ ID NO: 13 - DNA, 20 bp, Artificial sequence
  Synthetic oligonucleotide
tggatgtgcggttcttccgt

SEQ ID NO: 14 - DNA, 20 bp, Artificial sequence
  Synthetic oligonucleotide
agccttatggcttatttaag

SEQ ID NO: 15 - DNA, 20 bp, Artificial sequence
  Synthetic oligonucleotide
cgagatttttgacgttttga

SEQ ID NO: 16 - DNA, 20 bp, Artificial sequence
  Synthetic oligonucleotide
attcttgcccatcggggcca

SEQ ID NO: 17 - DNA, 20 bp, Artificial sequence
  Synthetic oligonucleotide
ttatggcttctgcgtgatac

SEQ ID NO: 18 - DNA, 20 bp, Artificial sequence
  Synthetic oligonucleotide
agagcccgcgtccgccatta

SEQ ID NO: 19 - DNA, 20 bp, Artificial sequence
  Synthetic oligonucleotide
cttaaactgagtgggtgttc

SEQ ID NO: 20 - DNA, 20 bp, Artificial sequence
  Synthetic oligonucleotide
tactgttgctgctgatcgtt

SEQ ID NO: 21 - DNA, 20 bp, Artificial sequence
  Synthetic oligonucleotide
ttgtgagttattgcactacc

SEQ ID NO: 22 - DNA, 20 bp, Artificial sequence
  Synthetic oligonucleotide
aacggggcgcttggtggatg

SEQ ID NO: 23 - DNA, 900 bp, Mus musculus
tgtttgctcgtttcccgtggatgtgcggttcttccgtggtttctctccatctaaggagctttgggggaacatttttagttcccctaccaccaagccttatggcttatttaagaaaacatatcaaaattccacgagatttttgacgttttgatatgttctggtaagattttttttttgacatgtcctccatactttttgatatttgtaatattttcagtcaatttttcatttttaaggaatatttctttgttgtgccttttggttgatacttgtgtgtgtatggtggacttacctttctttcattgtttatatattcttgcccatcggggccacggatacctgtgtgtcctccccgccattccatgcccaacggggttttggatacttacctgccttttcattctttttttttcttattatttttttttctaaacttgcccatctgggctgtggatacctgcttttattctttttttcttctccttagcccatcggggccatggatacctgctttttgtaaaaaaaaaaaaaaaaacaaaaaaacctttctcggtccatcgggacctcggatacctgcgtttagtctttttttcccatgcccaacggggcctcggatacctgctgttattatttttttttctttttcttttgcccatcggggctgtggatacctgctttaaattttttttttcacggcccaacggggcgcttggtggatggaaatatggttttgtgagttattgcactacctggaatatctatgcctcttatttgcgtgtactgttgctgctgatcgtttggtgctgtgtgagtgaacctatggcttagaaaaacgactttgctcttaaactgagtgggtgttcagggcgtggagagcccgcgtccgccattatggcttct

SEQ ID NO: 24 - DNA, 892 bp, Rattus norvegicus
tatttgctcgtctcccgtggatgtgaggtttcctccgtggtttctctccatctaaagggcttttggggaacatttttaatccccctaccaccatgccttatggtgtatttaagaaaacatatcaaaattacataagatttttgatgttttgatatgttctcgtaaggttttcttgacacgtcctccatattttttgatatttgtaatatttttggtctatttttcatttctaaggagtatttgtctgtcgtgcattttagttgacagctgtgtgtggtggacttacctttctttctttaactgtttttacattttttttgcccatcggggccacggatacctgtgtgtcctcccagccattccatgtccagctgggcttgggatacttaacctgccttttaatccttttttcttcttcttacttttcttcttctaaacttgcccatctgggttgtggatacctgcttttattctttttttcttcttctccttagcccatcggggccatggatacctgctttttaccaaaaaacgccgtatttctcggtccatcgggacctcggatacctgcgtttagtttttttcccatgcccaacggggcctcggatacctgctttaatttttttttcttttccttttgcccatcggggctgtggatacctgctttaattttttttttcacggcccatcggggcatttggtggatggaaataatggttttgtgagttattgaactacctggaatatatctatgcctttttatttccgtttgctgttggtgctgatcgtttggctgtgtgagtgaacctatggcttagaaaaacgacttggcgattaagcctagtgggtgttcagggcgtggagaacccgtgtccgccatcttacggtttct

SEQ ID NO: 25 - DNA, 923 bp, Felis catus
tatttcttcctttcccggggtggaagcttgctaacagtggatatctttgcccgtgtggttctttctggaacattttccagccccgaccactccttatggcgtatttcttttaaaaaattcacgaaaattccataaaatattttaacaattctaaactttctccgagtgttctcttgacatctcctccctattttttcaggtatttggaatatttttaggtaattttccattttaaaggaatttttcactggaatggtttttggttgatgcctctgctttgtcgtggtttagtttttttccccccttctcttttctacattttgcccatcggggctgtggataccaggttttattatcatttttttcgcccaacggggctgtggatacctgcgttttaattcttttcttttatttattttttttaatttgcccatcggggcagcggatacctgcttttaattttttttttttttcacccttagcccatcggggcctcggatacctgctgtgtctccttccctccccttaaccctctggcccatcggggcaatggataccagctttaaaaaaagttcctttttggcccatcggggcctcggatacctgcttttattattttttttcccttgcccatcggggccttggatacctgctttatttatttattttttttcctttgcccatcggggctgtggatacctgcttagatttttttttttctcatggcccatcggggccttttatggatggaaatgttggcttttgtggttcgttgtactgtctggaatgtctacaaatttatgctgctaatcaatcgtttggtgttgtgtgagtggacctacggctttagctgggagatgacttagcagttaggccaaggagttaggctggggaggaaagatggcggccacttcagccgcttg

SEQ ID NO: 26 - DNA, 900 bp, Homo sapiens
tatttcttactctctcggggctggaagcttcctgactgaagatctctctgcacttggggttctttctagaacattttctagtcccccaacaccctttatggcgtatttctttaaaaaaatcacctaaattccataaaatatttttttaaattctatactttctcctagtgtcttcttgacacgtcctccatatttttttaaagaaagtatttggaatattttgaggcaatttttaatatttaaggaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttggttttgtgggttgttgcactctctggaatatctacacttttttttgctgctgatcatttggtggtgtgtgagtgtacctaccgctttggcagagaatgactctgcagttaagctaagggcgtgttcagattgtggaggaaaagtggccgccattttag

SEQ ID NO: 27 - DNA, 9639 bp, Artificial sequence
  Synthetic oligonucleotide
  misc_feature (1)..(9639): n is any base
accagaagagggcatcagatgtcattacgggtggttgtgagccaccatgtggttgctgggatttgaacttcggaccttcggaagagcagtcgggtgctcttacccactgagccatctcaccagcccaacttctccctttcttaatcctaccattcacactctcccttacaaacttcttgcctagatttcttcaacaatgcatgcaaaagatttttaagcagataactattagatcagaggcctacatcctgtttagtctttgatggtgaagatcaagtttgcttcatgtccttccatacatcaggaaatagtttaaagataacaccttctcctttcccaacccttgtttgttcattaataccaaaaaaaaaaaaaaaaaaaaaagtggggaacacagggttaaaaaaaattctgaattcaaaaaacccactaatccttgagctcaccacatggtcagaacttgaagattgaacctctgtacatgctatcacactgacttcatatgtgcatcagtattgttatgtttagatttccctggagtcattcattccccctgaatctttaggattcttagaatctttccacttcttcacatggagaagctttgggtaaagacataccttttaggtctgaatgctccaaaatttctcacattattgttaagttgtggattaattgttagtttccatttactgtaagaaaattctctgataatagctgaatgatgcactaatctatggatataaaaacaaaaatattagaagtaactggattgctgtttctttagcagaacaccagaaggcgcgccaaaaaagcaccgactcggtgccacttttcaagttgataacggactagccttattttaacttgctatttctagctctaaaacnnnnnnnnnnnnnnnnnnnnaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatctatctgagacggaaaaaagcaccgactcggtgccacttttcaagttgataacggactagccttattttaacttgctatttctagctctaaaacnnnnnnnnnnnnnnnnnnnnaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatctatctgagacggaaaaaagcaccgactcggtgccacttttcaagttgataacggactagccttattttaacttgctatttctagctctaaaacnnnnnnnnnnnnnnnnnnnnaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatctatctgagacgggctagcggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaatcgtacggccaccatgcccaagaaaaaacgacgggttggaaccactgagaagaaatactccatcggcctggacatcggaaccaacagcgtgggctgggccgtgatcaccgacgactataaggtgcccagcaagaagttcaaagtgctgggcaacacaaacagaaagagcatcaagaagaacctgatgggcgccctgctgttcgactccggagagaccgccgaagcaacccggctgaagcgcaccgccagaagacggtacaccagaagaaaaaacaggatcagatacctgcaggagatcttcgccaacgagatggccaaactggacgatagctttttccagcggctggaagaatccttcctggtggaggaggacaagaaaaacgagagacaccctatctttgggaacctggccgacgaggtggcctaccacagaaactaccccaccatctaccacctgagaaagaagctggccgactcccccgaaaaggccgacctgagactgatctacctcgccctggcccacatcatcaagtttcggggccacttcctgattgaaggcaagctgaacgccgagaactccgacgtggccaagctgttctaccagctgatccagacctacaatcagctgtttgaggaaagccctctggacgaaatcgaagtggacgccaaagggatcctgtccgcccggctgagcaagtctaagcggctggagaagctgattgccgtcttccccaacgagaagaaaaacggcctgttcggcaacattatcgccctggccctgggcctgacccccaactttaagagcaatttcgatctgaccgaggatgccaagctgcagctgtccaaggatacctacgacgacgatctggacgagctgctgggacagatcggagaccagtacgccgacctgttcagcgccgccaaaaacctgtccgatgccatcctgctgagcgacatcctgcggagcaacagcgaagtcaccaaagcgcccctgagcgccagcatggtgaagcggtacgacgagcaccaccaggacctggcgctgctgaagacactggtgagacagcagttccccgagaagtacgccgagatcttcaaggacgataccaaaaatggctacgctggctacgtgggaatcggcatcaagcaccggaagcggacaaccaagctggccacccaggaggagttttacaagttcatcaagccgatcctggaaaagatggatggagccgaagagctgctggccaagctgaaccgggacgacctgctgagaaagcaacggaccttcgacaacggatccatccctcaccagatccacctgaaagagctgcacgctatcctgagacggcaagaagaattttaccccttcctgaaggagaaccgggagaagatcgaaaagatcctgaccttcagaatcccctactacgtggggccactggcccggggcaatagcagattcgcctggctgacccggaagtctgaagaggccatcaccccctggaattttgaggaggtggtggacaaaggcgccagcgcccagtccttcatcgagcggatgaccaacttcgatgagcagctgccaaacaagaaggtcctgcccaagcactctctgctgtacgaatattttaccgtgtacaacgaactgaccaaagtgaagtacgtgactgagagaatgcggaagcctgaattcctgtccggagagcagaagaaagccatcgtggacctgctgttcaagaccaacagaaaagtgaccgtgaaacagctgaaggaggactacttcaagaagatcgagtgcttcgacagcgtggagattatcggagtggaagacagattcaacgccagcctgggcacctaccacgatctgctgaagatcatcaaggataaggacttcctggacaacgaggagaacgaagacatcctggaggacatcgtgctgacactgaccctgttcgaggaccgggaaatgatcgaagaacggctcaaaacatacgctcacctgttcgatgacaaggtgatgaagcagctgaagagaagacactacaccggctggggccggctgtcccggaagatgatcaacggaatccgggacaagcagtccggcaagacaatcctggacttcctgaagtccgacggcttcagcaacagaaacttcatgcagctgatccacgacgacagcctgacctttaaagaggagatcgagaaagcccaggtgtccggccaaggagactctctgcacgagcagatcgccgacctggccggcagccccgccatcaaaaaaggcattctgcagaccgtgaagatcgtggatgagctggtgaaagtgatgggccacaagcctgagaacatcgtgatcgagatggccagagagaaccagaccaccaccaaaggactgcagcagtccagggagcggaaaaagcggatcgaggaaggcatcaaggagctggaaagccagattctgaaggaaaaccccgtggaaaacacccagctgcagaacgaaaagctgtacctgtactacctgcagaatggcagagatatgtacgtggatcaggagctggatatcaatcggctgtccgactacgatgtggaccacatcgtgccccagagcttcatcaaggacgactccatcgataacaaagtgctgacccggtctgtggagaacagaggcaagagcgacaacgtgcccagcgaagaagtggtgaagaagatgaagaactactggcggcagctgctgaacgccaagctgatcacccagagaaagttcgacaacctgaccaaggccgagcggggcggcctgagcgaagccgataaggctggcttcatcaagcggcagctggtggaaaccaggcagattacaaagcacgtggcccggatcctggacagcagaatgaacaccaagagagacaagaacgacaagcccatccgggaggtgaaggtgatcaccctgaagagcaaactggtgagcgacttcagaaaagacttccagctgtacaaagtgcgggacatcaacaactaccaccacgcccacgacgcctacctgaacgccgtggtgggcaccgctctgatcaagaagtaccccaagctggagagcgagttcgtgtacggcgattataaggtgtacgatgtgcggaaaatgatcgccaagagcgagcaggagatcggcaaggccaccgccaaacggttcttctacagcaacatcatgaatttcttcaagaccgaggtgaagctggccaacggcgagatcagaaagagacctctgattgaaaccaacggcgagaccggcgaagtggtgtggaacaaggagaaggacttcgccaccgtgcggaaggtgctggccatgccccaggtgaacatcgtgaagaagaccgaggtgcagactggcgggttctctaaggaatctatcctgagcaagcgggagagcgccaaactgatcccccggaagaagggctgggacaccagaaagtacggcggattcggcagcccaaccgtggcctacagcatcctggtggtggccaaggtggagaagggcaaggccaaaaagctgaaaagcgtgaaagtgctggtgggaatcaccatcatggaaaagggcagctacgagaaggaccccattggattcctggaagcaaagggctacaaggacatcaagaaggagctgatctttaaactgccgaagtacagcctgttcgaactggagaacggccggagacggatgctggccagcgccaaggagctgcagaaggccaatgagctggtgctgccccagcacctggtgagactgctgtactacacccagaacatcagcgccaccaccggcagcaacaacctgggctatatcgagcagcaccgggaggaattcaaagagatcttcgaaaagatcatcgatttcagcgagaagtacatcctgaagaacaaggtgaacagcaacctgaagagcagtttcgatgaacagttcgcagtcagcgactccatcctgctgagcaattccttcgtgtccctgctgaaatacacctccttcggagcctccggcggcttcaccttcctggacctggacgtgaagcagggccggctgcggtaccagaccgtgaccgaagtgctggatgccaccctgatctaccagtccatcaccggcctgtacgagacccggaccgacctgagccagctgggcggagaccccaagaaaaaacgacgggttggaaccacttagacgcgtgcgcaccgcatctgcgttcgcgctctctctcctcaggacgacaatcgacaatcgtgctgtggttgcagggtggccccgtcctccgccgtggctccatcgccgccactgcagccggcgcctgtttttgacgtgtacatagattgactcgttttacctcattttgttatttttcaacaaagccctgtggaaaggaaatggaaaacttgaagcattaaagccagccattctgttttgctccaatctcgagccgtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttnnnnnnnnnnnnnnnnnnnngttttagagctagaaatagcaagttaaaataaggctagtccgttatcaacttgaaaagtggcaccgagtcggtgcttttttccgtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttnnnnnnnnnnnnnnnnnnnngttttagagctagaaatagcaagttaaaataaggctagtccgttatcaacttgaaaagtggcaccgagtcggtgcttttttccgtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttnnnnnnnnnnnnnnnnnnnngttttagagctagaaatagcaagttaaaataaggctagtccgttatcaacttgaaaagtggcaccgagtcggtgcttttttgcggccgcttgagcctctctagtctcatgttcttgagggggaaacctttaatcccagcacttgggaggcagaggaaggcggatttctgagttcgaggccagcctggtaaacagattgagttccaggacagccaggatttatgcagagaaaccctgtctcaaaaaagtgtggtaaatgcacaaatgtgcaatctgagagtataggagcatgatagctacatgcaaccagcctcttctatagagtcctaaaacactccatgttccacagcaaggctatgtagaacactaaaatgtgggttgatgagttagctttcgtaggtaaaggtacttgttaacagtgtgaggcttttgaggttgatcattagggcacaaactgtagaaaacttcccccaataaataaagttaaagaaaatctagagacagaatacaggacagtgagtataggtaaactacagaaatggtggtagtactggccctggggtagtactgggccttttaatttaatcttagcacc

SEQ ID NO: 28 - DNA, 7772 bp, Artificial sequence
  Synthetic oligonucleotide
  misc_feature (1)..(7772): n is any base
accagaagagggcatcagatgtcattacgggtggttgtgagccaccatgtggttgctgggatttgaacttcggaccttcggaagagcagtcgggtgctcttacccactgagccatctcaccagcccaacttctccctttcttaatcctaccattcacactctcccttacaaacttcttgcctagatttcttcaacaatgcatgcaaaagatttttaagcagataactattagatcagaggcctacatcctgtttagtctttgatggtgaagatcaagtttgcttcatgtccttccatacatcaggaaatagtttaaagataacaccttctcctttcccaacccttgtttgttcattaataccaaaaaaaaaaaaaaaaaaaaaagtggggaacacagggttaaaaaaaattctgaattcaaaaaacccactaatccttgagctcaccacatggtcagaacttgaagattgaacctctgtacatgctatcacactgacttcatatgtgcatcagtattgttatgtttagatttccctggagtcattcattccccctgaatctttaggattcttagaatctttccacttcttcacatggagaagctttgggtaaagacataccttttaggtctgaatgctccaaaatttctcacattattgttaagttgtggattaattgttagtttccatttactgtaagaaaattctctgataatagctgaatgatgcactaatctatggatataaaaacaaaaatattagaagtaactggattgctgtttctttagcagaacaccagaaggcgcgccaaaaaannnnnnnnnnnnnnnnnnnnnnnatctacacttagtagaaattannnnnnnnnnnnnnnnnnnnnnnatctacacttagtagaaattannnnnnnnnnnnnnnnnnnnnnnatctacacttagtagaaattannnnnnnnnnnnnnnnnnnnnnnatctacacttagtagaaattannnnnnnnnnnnnnnnnnnnnnnatctacacttagtagaaattacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatctatctgagacgggctagcggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaatcgtacggccaccatggccccaaagaagaagcggaaggtcggtatccacggagtcccagcagccagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgcgcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaagggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcaccccaagaaaaaacgacgggttggaaccacttagacgcgtgcgcaccgcatctgcgttcgcgctctctctcctcaggacgacaatcgacaatcgtgctgtggttgcagggtggccccgtcctccgccgtggctccatcgccgccactgcagccggcgcctgtttttgacgtgtacatagattgactcgttttacctcattttgttatttttcaacaaagccctgtggaaaggaaatggaaaacttgaagcattaaagccagccattctgttttgctccaatctcgagccgtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtaatttctactaagtgtagatnnnnnnnnnnnnnnnnnnnnnnntaatttctactaagtgtagatnnnnnnnnnnnnnnnnnnnnnnntaatttctactaagtgtagatnnnnnnnnnnnnnnnnnnnnnnntaatttctactaagtgtagatnnnnnnnnnnnnnnnnnnnnnnntaatttctactaagtgtagatnnnnnnnnnnnnnnnnnnnnnnnttttttgcggccgcttgagcctctctagtctcatgttcttgagggggaaacctttaatcccagcacttgggaggcagaggaaggcggatttctgagttcgaggccagcctggtaaacagattgagttccaggacagccaggatttatgcagagaaaccctgtctcaaaaaagtgtggtaaatgcacaaatgtgcaatctgagagtataggagcatgatagctacatgcaaccagcctcttctatagagtcctaaaacactccatgttccacagcaaggctatgtagaacactaaaatgtgggttgatgagttagctttcgtaggtaaaggtacttgttaacagtgtgaggcttttgaggttgatcattagggcacaaactgtagaaaacttcccccaataaataaagttaaagaaaatctagagacagaatacaggacagtgagtataggtaaactacagaaatggtggtagtactggccctggggtagtactgggccttttaatttaatcttagcacc

(19) United States
(12) Patent Application Publication
(10) Pub. No.: US 2024/0011002 A1
(43) Pub. Date: Jan. 11, 2024

(54) DAUGHTERLESS MALE MAMMALS FOR NON-HUMAN POPULATION SUPPRESSION

(71) Applicant: Massachusetts Institute of Technology, Cambridge, MA (US)

(72) Inventor: Kevin Michael ESVELT, Cambridge, MA (US)

(73) Assignee: Massachusetts Institute of Technology, Cambridge, MA (US)

(21) Appl. No.: 17/766,762

(22) PCT Filed: Oct. 8, 2020
(86) PCT No.: PCT/US2020/054787

§ 371 Date: Apr. 6, 2022

Related U.S. Application Data
(60) Provisional application No. 62/912,323, filed on Oct. 8, 2019.

(51) Int. Cl. C12N 9/22; A01K 67/027; C12N 15/90

(52) U.S. Cl. CPC C12N 9/22; A01K 67/0276; C12N 15/907; A01K 2217/075; C12N 2310/20; A01K 2227/105

(57) ABSTRACT

The invention relates, in part, to methods of preparing male organisms of a species that include engineering the male organisms in a manner that eliminates post-embryonic viability of the female offspring and female descendants of the engineered male organisms. Certain aspects of the invention include use of such prepared engineered male organisms in methods of controlling population levels of organisms of the species.

### RELATED APPLICATIONS

This application claims the benefit under 35 U.S.C. § 119(e) of U.S. Provisional application Ser. No. 62/912,323 filed Oct. 8, 2019, the disclosure of which is incorporated by reference herein in its entirety.

### FIELD OF THE INVENTION

The invention relates, in part, to methods of preparing and using engineered male organisms that are reproductively fit, but whose female descendants are non-viable beyond an embryonic stage of development.

### BACKGROUND OF THE INVENTION

Existing and growing populations of mammalian pest organisms, such as rodents, swine, etc. result in economic losses and health and safety concerns around the world. Currently, efficient and effective options to reduce and/or control rodent and other pest organism populations are lacking. A general concept of using daughterless males for population suppression was postulated by A. Burt and A. Deredec (2018) Proc. R. Soc. B 285:20180776 but neither a workable approach nor methods of constructing such a daughterless male organism for effective population control have been identified. Methods to produce daughterless male organisms adequate for successful population suppression and the ability to achieve a robust male-linked daughterless phenotype in multiple species of mammals remain unknown and unavailable.

### SUMMARY OF THE INVENTION

According to an aspect of the invention, a composition is provide that includes a sequence encoding a DNA nuclease programmed to target a preselected X-chromosomal gene. In some embodiments, the DNA nuclease is a Cas9 nuclease. In some embodiments, the DNA nuclease is a Cas12a nuclease. In certain embodiments, if in a cell, the DNA nuclease is constitutively expressed. In some embodiments, if expressed in a cell, the DNA nuclease is capable of disrupting an activity of a preselected X-chromosomal gene. In some embodiments, the preselected X-chromosomal female-specific non-coding RNA gene is an Xist gene. In some embodiments, the composition also includes one or more promoter-encoding sequences capable of directing activity of the DNA nuclease to the preselected X-chromosomal female-specific non-coding RNA gene. In certain embodiments, the promoter-encoding sequence is located at one or both of upstream or downstream of the sequence encoding the DNA nuclease. In certain embodiments, the encoded promoter is a constitutive promoter. In some embodiments, the encoded promoter is a polymerase III promoter. In some embodiments, the encoded promoter is a U6, 7SK, or tRNA promoter. In some embodiments, the composition also includes one or more sequences each encoding a preselected guide RNA, wherein when expressed in a cell the preselected guide RNAs are capable of editing DNA of a preselected X chromosomal female-specific non-coding RNA gene. In certain embodiments, the sequence encoding the preselected guide RNA encodes a guide RNA sequence selected from SEQ ID NOs: 11-22. In some embodiments, the composition also includes a preselected endogenous gene, wherein the preselected endogenous gene is expressed in the germline of males of the organism. In some embodiments, the one or more of the preselected guide RNAs is encoded within one or more introns of the encoded DNA nuclease. In certain embodiments, the preselected endogenous gene is a Y chromosomal gene. In some embodiments, the Y chromosomal gene is a Zfy, Ddx3y, or Eif2s3y gene. In some embodiments, the composition also includes a 2A-sequence linking the nuclease to the preselected endogenous gene in a translational fusion. In some embodiments, the gene altered by the 2A-sequence comprises a modified Kozak sequence. In certain embodiments, the composition also includes an IRES-sequence linking the nuclease to the preselected endogenous gene in a transcriptional fusion.

According to an aspect of the invention, a vector is provided that includes an embodiment of any aforementioned composition.

According to an aspect of the invention, a cell is provided that includes an embodiment of any aforementioned vector.

According to an aspect of the invention, a cell is provided that includes an embodiment of any aforementioned composition. In some embodiments, the cell is a germline cell. In some embodiments, the cell is a non-human mammalian cell. In certain embodiments, if the cell comprises the expressed nuclease, the nuclease is capable of disrupting an activity of a preselected X-chromosomal female-specific non-coding RNA gene in the cell. In certain embodiments, the preselected X-chromosomal female-specific non-coding RNA gene is an Xist gene.

According to an aspect of the invention, an engineered gene is provided that includes an embodiment of any aforementioned composition. In some embodiments, the engineered gene is a Y chromosomal engineered gene.

According to an aspect of the invention, a method of altering organisms of a species is provided, the method including: (a) engineering a male organism of a species, wherein an activity of an X chromosome non-coding RNA gene is disrupted in female descendants of the engineered male organism; and (b) producing one or more descendant organisms from the engineered male organism and a female organism of the species, wherein the disruption of the activity of the X chromosome non-coding RNA gene in the female descendants is embryonically lethal to the female descendants. In some embodiments, a means of producing the descendant organisms includes impregnating a female organism of the species with genetic material of the engineered male. In some embodiments, a means of impregnation includes a mating, artificial insemination, or an in vitro fertilization (IVF) method. In certain embodiments, the method also includes releasing the engineered male organism into a target population of organisms of the species. In some embodiments, a means for the disrupting of an activity of an X chromosome non-coding RNA gene in female descendants of the engineered male organism comprises encoding on the engineered male organism's Y chromosome a nuclease capable of disrupting the activity of the X chromosome non-coding RNA gene. In some embodiments, the nuclease is a Cas9 or a Cas12a nuclease. In some embodiments, the nuclease is an RNA-guided nuclease. In certain embodiments, one or more preselected guide RNAs are encoded within one or more introns of the encoded nuclease. In some embodiments, the nuclease is capable of cutting on both sides of one or more stem-loop repeats in the X chromosome non-coding RNA gene. In certain embodiments, the nuclease is capable of cutting within a stretch of stem-loop repeats. In some embodiments, the nuclease is encoded in an endogenous gene in the engineered male organism. In some embodiments, the endogenous gene is expressed in the germline of males of the organism. In some embodiments, the endogenous gene is a Y-chromosomal gene. In some embodiments, the endogenous gene is Eif2s3y, Zfy, or Ddx3y. In certain embodiments, the method also includes inserting a plurality of introns into the coding region of the endogenous gene, wherein the inserted plurality of introns increases a level of translation of the endogenous gene compared a level of translation of the endogenous gene in the absence of the inserted plurality of introns. In some embodiments, the method also includes an N-terminal 2A-peptide fusion to the endogenous gene. In some embodiments, the N-terminal 2A-fusion to the endogenous gene comprises a modified Kozak sequence, wherein the modified Kozak sequence increases a level of translation of the endogenous gene compared to a level of translation of the endogenous gene in the absence of the modified Kozak sequence. In some embodiments, the method also includes one or both of: (a) inserting a plurality of introns into the coding region of the endogenous gene and (b) inserting a modified Kozak sequence into the N-terminal 2A fusion protein, wherein one or both of (a) and (b) increases a level of translation of the endogenous gene compared a level of translation of the endogenous gene in the absence of (a), (b), or both (a) and (b), respectively. In some embodiments, one or more introns in the plurality introns is a synthetic intron. In certain embodiments, one or more of the introns in the plurality of introns is native to a gene of the organisms of the species and is included in a sequence encoding the nuclease. In certain embodiments, the method also includes encoding a polymerase III promoter upstream of the one or more encoded preselected guide RNAs, wherein the polymerase III enhances the expression of the one or more preselected guide RNAs compared to the expression of the one or more preselected guide RNAs in the absence of the encoded polymerase III promoter. In some embodiments, the polymerase III promoter is a U6 promoter, a 7SK promoter, an H1 promoter, or a tRNA promoter. In some embodiments, the polymerase III promoter is a synthetic promoter. In certain embodiments, the polymerase III promoter is a polymerase III promoter native to the organism species. In some embodiments, the polymerase III promoter is a polymerase III promoter not native to the organism species. In some embodiments, the engineered male organism is a cisgenic engineered male organism. In certain embodiments, the X chromosome non-coding RNA gene is an X-inactive specific transcript (Xist) gene. In certain embodiments, the disruption of the Xist gene comprises deletion of at least a portion of the Xist gene. In some embodiments, a means for the disruption of the Xist gene comprises a method comprising use of one or more sgRNA set forth as SEQ ID NOs: 11-22. In certain embodiments, the disruption of the Xist gene comprises deletion of one, two, three, or more stem-loop repeats in Xist region A within exon 1 of the Xist gene. In certain embodiments, the engineered male organism is born in a wild population of organisms of the species. In some embodiments, the engineered male organism is born in captivity. In some embodiments, the female organism is a captive female organism. In some embodiments, the female organism is obtained from a wild population of organisms of the species. In some embodiments, the female organism is obtained from the target population of organisms. In certain embodiments, the target population is a captive population of organisms of the species. In certain embodiments, the organism is a mammal. In some embodiments, the organism is of the genus Rattus, Mus, Sus, Felis or Canis. In some embodiments, the organism is not a human. In certain embodiments, the disrupted activity of the X chromosome non-coding RNA gene in a female embryo of a female impregnated by the engineered male organism or a descendant of the engineered male organism is lethal to the female embryo.

According to another aspect of the invention, a method of increasing translation an N-terminal 2A-fusion to a gene is provided, the method including: preparing an N-terminal 2A-fusion to a gene, wherein the N-terminal 2A-fusion to the gene comprises a modified Kozak sequence, wherein the modified Kozak sequence increases a level of translation of the gene compared to a level of translation of the gene in the absence of the modified Kozak sequence. In some embodiments, the method also includes encoding a nuclease in the N-terminal 2A-fusion. In some embodiments, the nuclease is a Cas9 or Cas12a nuclease. In certain embodiments, the gene is in an organism. In certain embodiments, the gene is an endogenous gene of the organism. In some embodiments, the endogenous gene is expressed in the germline of males of the organism. In some embodiments, the endogenous gene is a Y-chromosomal gene. In certain embodiments, the gene is Eif2s3y, Zfy, or Ddx3y.

According to another aspect of the invention, a method of increasing translation of an N-terminal 2A-fusion to a gene is provided, the method including: preparing an N-terminal 2A-fusion to a gene, wherein the preparing comprises inserting a plurality of introns into the coding region of the gene, wherein the inserted plurality of introns increases a level of translation of the gene compared to a level of translation of the gene in the absence of the inserted plurality of introns. In some embodiments, the method also includes encoding a nuclease in the N-terminal 2A-fusion. In certain embodiments, the nuclease is a Cas9 or Cas12a nuclease. In certain embodiments, the gene is in an organism. In some embodiments, the gene is an endogenous gene of the organism. In some embodiments, the endogenous gene is expressed in the germline of males of the organism. In certain embodiments, the endogenous gene is a Y-chromosome gene. In certain embodiments, the gene is Eif2s3y, Zfy, or Ddx3y. In some embodiments, one or more introns in the plurality introns is a synthetic intron. In some embodiments, one or more of the introns in the plurality of introns is native to a gene of the organisms of the species and is included in a sequence encoding the nuclease.

According to another aspect of the invention, a method of engineering a cisgenic organism is provided, the method including: (a) deriving DNA fragments from one or more of: (i) an organism of a species of interest; (ii) a prokaryotic cell in close ecological association with the species of interest; (iii) a eukaryotic cell in close ecological association with the species of interest; and (b) introducing the derived DNA fragments into one or more organisms of the species of interest, wherein all of the introduced DNA fragments are the derived DNA fragments. In some embodiments, the organism of the species of interest is a multicellular eukaryotic organism. In certain embodiments, the introduced derived DNA fragments are obtained from a cell of a donor organism of the species of interest. In certain embodiments, the introduced derived DNA fragments are obtained from a cell present within the multicellular eukaryotic organism's commensal microbiota. In some embodiments, the introduced DNA fragments are at least 15, 16, 17, 18, 19, 20, 21, 22, 23, 24, 25, 26, 27, 28, 29, 30, or more base pairs in length. In certain embodiments, the method also includes: splicing together two, three, four, or more of the derived DNA fragments into a spliced derived DNA molecule, wherein one or more of the derived DNA fragments introduced are introduced as part of the spliced derived DNA molecule.

According to another aspect of the invention, a cisgenic engineered cell; is provided, the engineered cell including: (a) a cell of a species of interest, comprising introduced DNA fragments derived from one or more of: (i) an organism of the species of interest, (ii) a prokaryotic cell in close ecological association with the species of interest, and (iii) a eukaryotic cell in close ecological association with the species of interest, wherein all of the introduced DNA fragments are the derived DNA fragments, and the cell is a cisgenic engineered cell. In some embodiments, the species of interest is a multicellular eukaryotic organism. In certain embodiments, the introduced derived DNA fragments are DNA fragments from a cell of a donor organism of the species of interest. In some embodiments, the introduced derived DNA fragments are DNA fragments from a cell present within the multicellular eukaryotic organism's commensal microbiota. In some embodiments, the introduced DNA fragments are at least 15, 16, 17, 18, 19, 20, 21, 22, 23, 24, 25, 26, 27, 28, 29, 30, or more base pairs in length. In some embodiments, two, three, four, or more of the derived DNA fragments are spliced together.

According to another aspect of the invention, an engineered cell is provided, the engineered cell including an engineered Y-chromosomal endogenous gene that when expressed disrupts an activity of an X-chromosomal female-specific non-coding RNA gene in the cell. In certain embodiments, a nuclease encoded on the Y chromosome is a nuclease capable of disrupting the activity of the X chromosome female-specific non-coding RNA gene. In some embodiments, the nuclease is a Cas9 or Cas12a nuclease. In some embodiments, the nuclease is an RNA-guided nuclease. In some embodiments, one or more preselected guide RNAs are encoded within one or more introns of the encoded nuclease. In certain embodiments, the nuclease is capable of cutting on both sides of one or more stem-loop repeats in the X chromosome female-specific non-coding RNA gene. In certain embodiments, the nuclease is capable of cutting within a stretch of stem-loop repeats. In certain embodiments, the nuclease is encoded in the Y-chromosomal endogenous gene. In some embodiments, the cell is a germline cell. In some embodiments, the endogenous gene is Eif2s3y, Zfy, or Ddx3y. In certain embodiments, the engineered cell also includes a plurality of introns inserted in a coding region of the endogenous gene, wherein the inserted plurality of introns increases a level of translation of the endogenous gene compared a level of translation of the endogenous gene in the absence of the inserted plurality of introns. In some embodiments, engineered cell also includes an N-terminal 2A-peptide fusion to the endogenous gene. In some embodiments, the N-terminal 2A-fusion to the endogenous gene comprises a modified Kozak sequence, wherein the modified Kozak sequence increases a level of translation of the endogenous gene compared to a level of translation of the endogenous gene in the absence of the modified Kozak sequence. In some embodiments, the engineered cell also includes one or both of: (a) plurality of introns inserted into the coding region of the endogenous gene and (b) a modified Kozak sequence in the N-terminal 2A fusion protein, wherein one or both of (a) and (b) increases a level of translation of the endogenous gene compared a level of translation of the endogenous gene in the absence of (a), (b), or both (a) and (b), respectively. In certain embodiments, one or more introns in the plurality introns is a synthetic intron. In certain embodiments, one or more of the introns in the plurality of introns is native to a gene of the organisms of the species of the cell and is included in a sequence encoding the nuclease. In some embodiments, the engineered cell also includes: a polymerase III promoter encoded upstream of the one or more encoded preselected guide RNAs, wherein the polymerase III enhances the expression of the one or more preselected guide RNAs compared to the expression of the one or more preselected guide RNAs in the absence of the encoded polymerase III promoter. In some embodiments, the polymerase III promoter is a U6 promoter, a 7SK promoter, an H1 promoter, or a tRNA promoter. In certain embodiments, the polymerase III promoter is a synthetic promoter, or is a polymerase III promoter native to an organism of the species of the cell species, or is a polymerase III promoter not native to the organism species. In some embodiments, the engineered cell is a cisgenic-engineered cell. In some embodiments, the X chromosome female-specific non-coding RNA gene is an X-inactive specific transcript (Xist) gene. In certain embodiments, the disruption of the Xist gene comprises deletion of at least a portion of the Xist gene. In some embodiments, the engineered cell is a cell of a mammalian species. In some embodiments, the species is of the genus Rattus, Mus, Sus, Felis or Canis.

According to another aspect of the invention, a method of preparing an engineered organism is provided the method including: impregnating a female organism of a species with an engineered cell of any aforementioned embodiment of an engineered cell, wherein the female organism and the cell are of the same species, and a descendant resulting from the impregnation is an engineered organism. In certain embodiments, the engineered organism is an engineered daughterless male organism. In certain embodiments, the engineered organism is a mammal.

According to another aspect of the invention, a composition is provided that includes a guide RNA molecule that has a sequence selected from SEQ ID NOs: 11-22.

According to another aspect of the invention, a cell is provided that includes an embodiment of any of the aforementioned compositions that include a guide RNA molecule. In certain embodiments, the cell is a germline cell.

### BRIEF DESCRIPTION OF THE DRAWINGS

FIG. 1A-B provides sequence information. FIG. 1A shows a sequence alignment of Xist exon 1 region A in mice (M musculus, SEQ ID NO: 1, top sequence), rats (R. norvegicus, SEQ ID NO: 2, second sequence from top), cats (F. catus, SEQ ID NO: 3, third sequence from top), and humans (H. sapiens, SEQ ID NO: 4, fourth sequence from top). The critical stem-loop structural repeats required for functionality are highlighted and boxed. Mice and rats harbor 7.5 paired stem-loop repeats, while cats and humans encode 8.5. In mice, 5.5 synthetic repeats are enough for Xist function, while four are insufficient [Wutz, A., et al., (2002) Nature Genetics 30 (2): 167-174]. Stem-loop structural repeats are shown in FIG. 1B as NNGYCCAUCGGGGYN (SEQ ID NO: 5) and NYGGAUACCUGNN (SEQ ID NO: 6).

FIG. 2A-C shows schematic diagrams of example Mus musculus Y chromosomes encoding daughterless systems and of a screening strategy for sgRNAs used for Xist deletion. FIG. 2A illustrates a means with which to generate a daughterless mouse by encoding a functional Cas9 CRISPR system on the Y chromosome such that it disrupted the function of the X-encoded Xist gene in the male germline. FIG. 2B illustrates a means with which to generate a daughterless mouse by encoding a functional Cas12a CRISPR system on the Y chromosome such that it disrupted the function of the X-encoded Xist gene in the male germline. For strategies shown in both 2A and 2B, Region A of the mouse Xist locus featuring numerous functionally important hairpin repeats was amplified by PCR and subjected to a TIDE assay and the evaluation pipeline. FIG. 2C illustrates a screening process for candidate pairs of sgRNAs to determine whether they are capable of efficient Xist deletion in Mus cells, in which GFP is not expressed unless the 900 base-pair Xist region, which contains in-frame stop codons, is deleted. Terminology: ScCas9 is Streptococcus canis Cas9: US is U6 promoter; Ddx3y is a Y chromosome gene; Xist is an X chromosome gene; crRNA is CRISPR RNA; and sgRNA is Single Guide RNA.

FIG. 3 provides an image of a gel showing results of PCR amplification of Xist region sequence. The results demonstrated guide 5g2 paired with guide 3g1 were more active than guide 5g1 paired with guide 3g1, as evidenced by the presence of the smaller band resulting from deletion of the intervening DNA in the population of cells.

FIG. 4 provides a bar graph showing Xist-targeting SpCas9 sgRNA efficiency in 3T3 cells. Plain bars show Relative Indel formation and striped bars show Raw Tide Value.

FIG. 5 is a diagram of a 9639 bp compositional DNA construct, referred to herein as: HA-mEF1a-Sc+mbA anti-Xist, which uses the Cas9 protein from Streptococcus canis (a native rodent commensal, making it eco-cisgenic). The prepared construct is linear, but is shown in the figure as circular, to permit the level of detail to be shown.

FIG. 6 is a diagram of a 7772 bp compositional DNA construct, referred to herein as: HA-mEF1a-LbCas12aRR-mbA anti-Xist, which uses a Cas12a protein. The prepared construct is linear, but is shown in the figure as circular, to permit the level of detail to be shown.

FIG. 7 is sequence of 9639 bp compositional DNA construct, referred to herein as: HA-mEF1a-Sc+mbA anti-Xist, which is diagrammed in FIG. 5.

FIG. 8 is sequence of 7772 bp compositional DNA construct, referred to herein as: HA-mEF1a-LbCas12aRR-mbA anti-Xist, which is diagrammed in FIG. 6.

Brief Description of the Sequences

SEQ ID NO: 1 is region of M. musculus Xist nucleic acid sequence in exon 1.
tcttgcccatcggggccacggatacctgtgtgtcctccccgccattccatgcccaacggggttttggatacttacctgccttttcattcttttt
ttttcttattatttttttttctaaacttgcccatctgggctgtggatacctgcttttattctttttttttctccttagcccatcggggccatggatacc
tgctttttgtaaaaaaaaaaaaaaaaacaaaaaaacctttctcggtccatcgggacctcggatacctgcgtttagtctttttttcccatgccc
aacggggcctcggatacctgctgttattatttttttttctttttcttttgcccatcggggctgtggatacctgctttaaattttttttttcacggccc
aacggggcgcttggtggatggaa.
SEQ ID NO: 2 is region of R. norvegicus Xist nucleic acid sequence in to exon 1.
ttttgcccatcggggccacggatacctgtgtgtcctcccagccattccatgtccagctgggcttgggatacttaacctgccttttaatccttt
tttcttcttcttacttttcttcttctaaacttgcccatctgggttgtggatacctgcttttattctttttttcttcttctccttagcccatcggggccatg
gatacctgctttttaccaaaaaacgccgtatttctcggtccatcgggacctcggatacctgcgtttagtttttttcccatgcccaacggggc
ctcggatacctgctttaatttttttttcttttccttttgcccatcggggctgtggatacctgctttaattttttttttcacggcccatcggggcatttg
gtggatggaa.
SEQ ID NO: 3 is region of F. catus Xist nucleic acid sequence in exon 1.
ttttgcccatcggggctgtggataccaggttttattatcatttttttcgcccaacggggctgtggatacctgcgttttaattcttttcttttatttatt
ttttttaatttgcccatcggggcagcggatacctgcttttaattttttttttttttcacccttagcccatcggggcctcggatacctgctgtgtctc
cttccctccccttaaccctctggcccatcggggcaatggataccagctttaaaaaaagttcctttttggcccatcggggcctcggatacct
gcttttattattttttttcccttgcccatcggggccttggatacctgctttatttatttattttttttcctttgcccatcggggctgtggatacctgctt
agatttttttttttctcatggcccatcggggccttttatggatggaa.
SEQ ID NO: 4 is region of H. sapiens Xist nucleic acid sequence in exon 1.
ttttgcccatcggggctgcggatacctggttttattattttttctttgcccaacggggccgtggatacctgccttttaattcttttttattcgccca
tcggggccgcggatacctgctttttatttttttttccttagcccatcggggtatcggatacctgctgattcccttcccctctgaacccccaaca
ctctggcccatcggggtgacggatatctgctttttaaaaattttctttttttggcccatcggggcttcggatacctgctttttttttttttatttttcc
ttgcccatcggggcctcggatacctgctttaatttttgtttttctggcccatcggggccgcggatacctgctttgatttttttttttcatcgccca
tcggtgctttttatggatgaaa.
SEQ ID NO: 5 is stem loop sequence: nngyccaucggggyn.
SEQ ID NO: 6 is stem loop sequence: nyggauaccugnn.
SEQ ID NO: 7 is: LLXXGDVENPGP.
SEQ ID NO: 8 is: LLXXGDVSNPGP.
SEQ ID NO: 9 is: LLXXXGDVENPGP.
SEQ ID NO: 10 is: LLXXGDVSNPGP.
SEQ ID NO: 11 is gctcgtttcccgtggatgtg.
SEQ ID NO: 12 is gtggatgtgcggttcttccg.
SEQ ID NO: 13 is tggatgtgcggttcttccgt.
SEQ ID NO: 14 is agccttatggcttatttaag.
SEQ ID NO: 15 is cgagatttttgacgttttga.
SEQ ID NO: 16 is attcttgcccatcggggcca.
SEQ ID NO: 17 is ttatggcttctgcgtgatac.
SEQ ID NO: 18 is agagcccgcgtccgccatta.
SEQ ID NO: 19 is cttaaactgagtgggtgttc.
SEQ ID NO: 20 is tactgttgctgctgatcgtt.
SEQ ID NO: 21 is ttgtgagttattgcactacc.
SEQ ID NO: 22 is aacggggcgcttggtggatg.
SEQ ID NO: 23 is nucleic acid sequence of exon 1 of M. musculus Xist gene.
tgtttgctcgtttcccgtggatgtgcggttcttccgtggtttctctccatctaaggagctttgggggaacatttttagttcccctaccaccaag
ccttatggcttatttaagaaaacatatcaaaattccacgagatttttgacgttttgatatgttctggtaagattttttttttgacatgtcctccatac
tttttgatatttgtaatattttcagtcaatttttcatttttaaggaatatttctttgttgtgccttttggttgatacttgtgtgtgtatggtggacttacc
tttctttcattgtttatatattcttgcccatcggggccacggatacctgtgtgtcctccccgccattccatgcccaacggggttttggatactta
cctgccttttcattctttttttttcttattatttttttttctaaacttgcccatctgggctgtggatacctgcttttattctttttttcttctccttagcc
catcggggccatggatacctgctttttgtaaaaaaaaaaaaaaaaacaaaaaaacctttctcggtccatcgggacctcggatacctgcgttta
gtctttttttcccatgcccaacggggcctcggatacctgctgttattatttttttttctttttcttttgcccatcggggctgtggatacctgctttaa
attttttttttcacggcccaacggggcgcttggtggatggaaatatggttttgtgagttattgcactacctggaatatctatgcctcttatttgc
gtgtactgttgctgctgatcgtttggtgctgtgtgagtgaacctatggcttagaaaaacgactttgctcttaaactgagtgggtgttcaggg
cgtggagagcccgcgtccgccattatggcttct.
SEQ ID NO: 24 is nucleic acid sequence of exon 1 of R. norvegicus Xist gtene:
tatttgctcgtctcccgtggatgtgaggtttcctccgtggtttctctccatctaaagggcttttggggaacatttttaatccccctaccaccat
gccttatggtgtatttaagaaaacatatcaaaattacataagatttttgatgttttgatatgttctcgtaaggttttcttgacacgtcctccatattt
tttgatatttgtaatatttttggtctatttttcatttctaaggagtatttgtctgtcgtgcattttagttgacagctgtgtgtggtggacttacctttctt
tctttaactgtttttacattttttttgcccatcggggccacggatacctgtgtgtcctcccagccattccatgtccagctgggcttgggatactt
aacctgccttttaatccttttttcttcttcttacttttcttcttctaaacttgcccatctgggttgtggatacctgcttttattctttttttcttcttctc
cttagcccatcggggccatggatacctgctttttaccaaaaaacgccgtatttctcggtccatcgggacctcggatacctgcgtttagtttttttc
ccatgcccaacggggcctcggatacctgctttaatttttttttcttttccttttgcccatcggggctgtggatacctgctttaattttttttttcacg
gcccatcggggcatttggtggatggaaataatggttttgtgagttattgaactacctggaatatatctatgcctttttatttccgtttgctgttg
gtgctgatcgtttggctgtgtgagtgaacctatggcttagaaaaacgacttggcgattaagcctagtgggtgttcagggcgtggagaac
ccgtgtccgccatcttacggtttct.
SEQ ID NO: 25 is nucleic acid sequence of exon 1 region of F. catus Xist gene:
tatttcttcctttcccggggtggaagcttgctaacagtggatatctttgcccgtgtggttctttctggaacattttccagccccgaccactcct
tatggcgtatttcttttaaaaaattcacgaaaattccataaaatattttaacaattctaaactttctccgagtgttctcttgacatctcctccctatt
ttttcaggtatttggaatatttttaggtaattttccattttaaaggaatttttcactggaatggtttttggttgatgcctctgctttgtcgtggtttagt
ttttttccccccttctcttttctacattttgcccatcggggctgtggataccaggttttattatcatttttttcgcccaacggggctgtggatacct
gcgttttaattcttttcttttatttattttttttaatttgcccatcggggcagcggatacctgcttttaattttttttttttttcacccttagcccatcgg
ggcctcggatacctgctgtgtctccttccctccccttaaccctctggcccatcggggcaatggataccagctttaaaaaaagttcctttttgg
cccatcggggcctcggatacctgcttttattattttttttcccttgcccatcggggccttggatacctgctttatttatttattttttttcctttgccc
atcggggctgtggatacctgcttagatttttttttttctcatggcccatcggggccttttatggatggaaatgttggcttttgtggttcgttgtac
tgtctggaatgtctacaaatttatgctgctaatcaatcgtttggtgttgtgtgagtggacctacggctttagctgggagatgacttagcagtt
aggccaaggagttaggctggggaggaaagatggcggccacttcagccgcttg.
SEQ ID NO: 26 is nucleic acid sequence of exon 1 of H. sapiens Xist gene:
tatttcttactctctcggggctggaagcttcctgactgaagatctctctgcacttggggttctttctagaacattttctagtcccccaacaccc
tttatggcgtatttctttaaaaaaatcacctaaattccataaaatatttttttaaattctatactttctcctagtgtcttcttgacacgtcctccatatt
tttttaaagaaagtatttggaatattttgaggcaatttttaatatttaaggaatttttctttggaatcatttttggttgacatctctgttttttgtggatc
agttttttactcttccactctcttttctatattttgcccatcggggctgcggatacctggttttattattttttctttgcccaacggggccgtggata
cctgccttttaattcttttttattcgcccatcggggccgcggatacctgctttttatttttttttccttagcccatcggggtatcggatacctgctg
attcccttcccctctgaacccccaacactctggcccatcggggtgacggatatctgctttttaaaaattttctttttttggcccatcggggctt
cggatacctgctttttttttttttatttttccttgcccatcggggcctcggatacctgctttaatttttgtttttctggcccatcggggccgcggat
acctgctttgatttttttttttcatcgcccatcggtgctttttatggatgaaaaaatgttggttttgtgggttgttgcactctctggaatatctacac
ttttttttgctgctgatcatttggtggtgtgtgagtgtacctaccgctttggcagagaatgactctgcagttaagctaagggcgtgttcagatt
gtggaggaaaagtggccgccattttag.

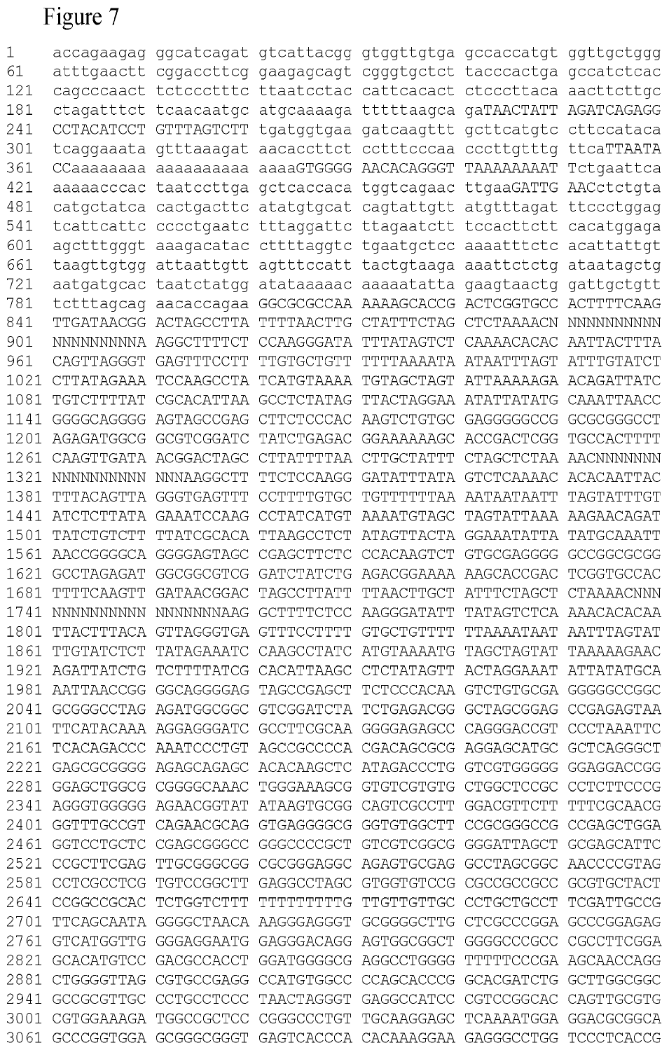

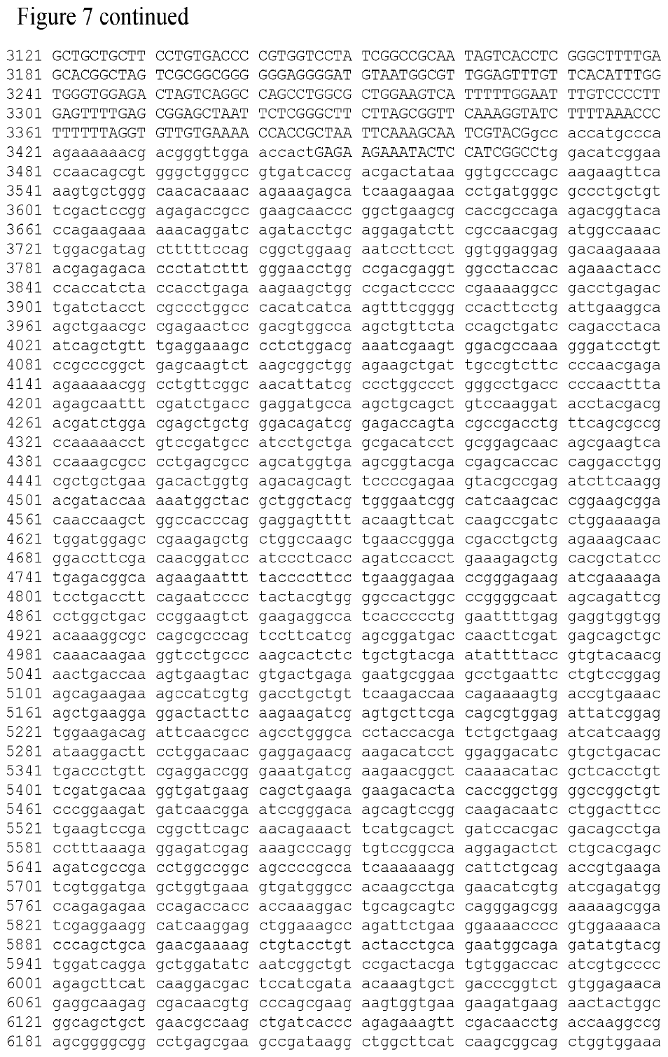

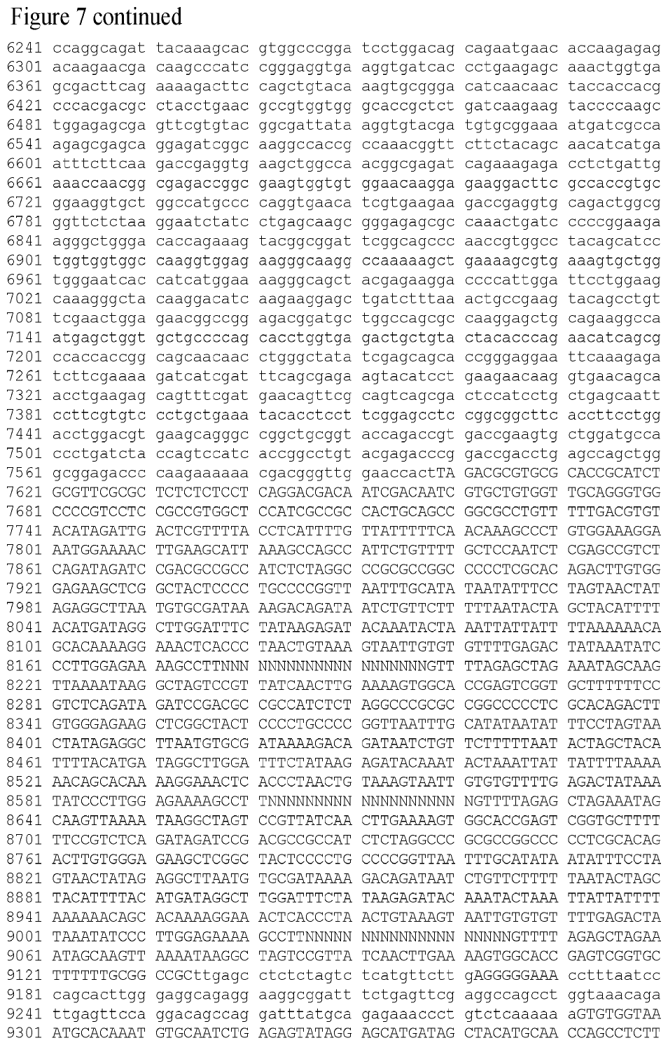

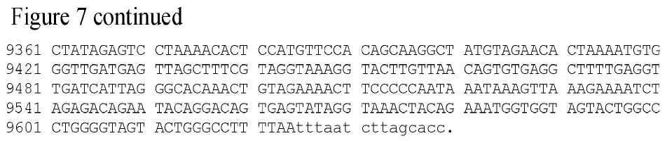

SEQ ID NO: 27 is sequence of 9639 bp compositional DNA construct (sequence shown in FIG. 7. Construct is referred to herein as: HA-mEF1a-Sc+mbA anti-Xist.

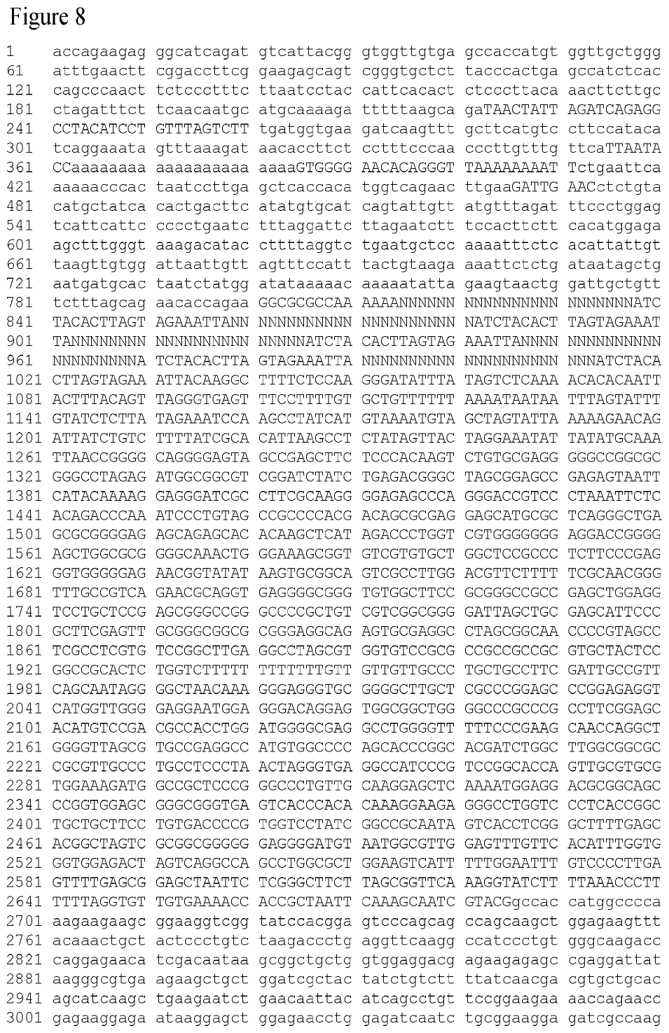

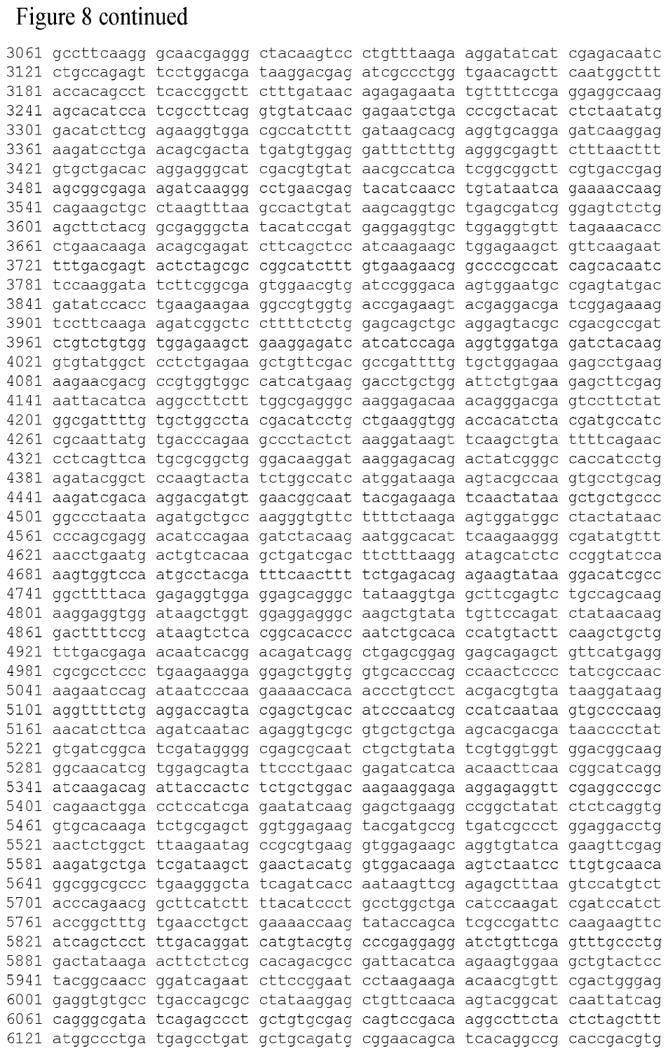

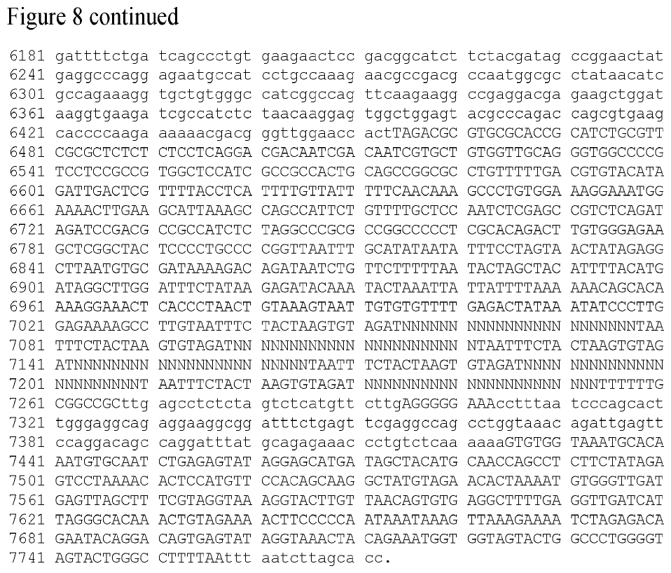

SEQ ID NO: 28 is sequence of 7772 bp compositional DNA construct (sequence shown in FIG. 8). Construct is referred to herein as: HA-mEF1a-LbCas12aRR-mbA anti-Xist.

### Detailed Description

Aspects of the invention, in part, include methods of preparing engineered daughterless male organisms and use of such prepared engineered daughterless male organisms in population suppression methods. Certain embodiments of the invention include methods of engineering male organisms, for example mammals, that are daughterless and produce male descendants that will themselves be daughterless. Certain aspects of the invention include introducing engineered daughterless male organisms into a target population of the organism, which can suppress the number of the organisms in that population for an extended period. This is, in part, because according to certain embodiments of the invention, the engineered daughterless male locus is not at a disadvantage relative to the competing wild-type male locus.

Certain embodiments of the invention include methods of engineering a male organism by encoding on the Y chromosome of the organism, a system that knocks out a critical sequence of an X-chromosome female-specific noncoding RNA gene, thereby disrupting the female-specific non-coding RNA gene's activity. Although inheriting the disrupted female-specific non-coding RNA gene may have little or no effect in male organism, inheriting a disrupted paternal female-specific non-coding RNA gene is embryonically lethal in female organisms. Engineered male organisms prepared as described herein are reproductively fit and capable of producing male descendants, but due to the engineered disruption of the activity of the X-chromosome female-specific non-coding RNA gene, no female descendants of the engineered male organism(s) are produced.

The term “system” as used herein in reference to knocking out a critical sequence of an X-chromosome female-specific noncoding RNA gene thereby eliminating its activity, means a gene editing system that is engineered into the genome of an organism of a species. In certain embodiments of the invention, a male organism is engineered to include a gene editing system, which in some embodiments of the invention is included in a germline cell of the male organism. In some embodiments of the invention, the gene editing system is included in germline cells, not other cells, of an engineered organism. Methods of the invention can be used to prepare an engineered male organism comprising a gene editing system that includes components located on the Y chromosome, that when expressed edit a sequence of an X-chromosomal female-specific non-coding RNA gene and disrupting its function. A gene editing system as set forth in methods of the invention is inherited by male descendants of the engineered male organism, and results in no female descendants of the engineered male organism(s).

In certain embodiments of the invention, a gene editing system comprises components positioned on a Y chromosome in an engineered organism. Certain embodiments of methods of the invention comprise encoding on the Y chromosome of an engineered male organism, one or more nucleases that when expressed function to disrupt an activity of a preselected X-chromosomal female-specific non-coding RNA gene. In addition, in certain embodiments of methods of the invention one or more guide RNAs are encoded on the Y chromosome of an engineered male organism. The encoded guide RNAs may, in some embodiments of the invention, be encoded within one or more introns of an encoded nuclease. The encoded guide RNAs may, in some embodiments of the invention, be encoded flanking the encoded nuclease. Components of the gene editing system are expressed, and in some embodiments of the invention, the resulting nuclease(s) and guide RNAs function to edit DNA of a preselected X chromosomal female-specific non-coding RNA gene in the engineered male organism. Male descendants of the engineered male organism inherit the engineered Y-chromosomal gene, which when expressed disrupts the X-chromosomal female-specific non-coding gene in the descendant male, which is embryonically lethal to females, which results in only male descendants.

Methods of the invention can be used to prepare engineered daughterless male organisms of many different mammalian species, regardless of substantial differences between species' genetics. It has now been identified that by engineering a male organism so an activity of an X-chromosome female-specific non-coding RNA gene is lost, impregnating a female organism with genetic material from the engineered male, results in a robust male-linked daughterless phenotype that can be used in population suppression methods. Although methods of the invention can be used for sustained population number suppression, methods of the invention do not include gene drive technology and therefore, engineered daughterless genes prepared and used in methods of the invention will not increase in frequency on their own due to superMendelian inheritance.

### Overview

In some instances, methods of the invention include engineering a male organism by encoding on the Y chromosome a system that knocks out the sequence of a female-specific non-coding RNA gene, which results in a lethal disruption of an activity of an X chromosome non-coding RNA gene in female descendants of the engineered male organism. One or more descendant organisms may be produced from the engineered male organism and a female organism of the species and the disruption of the activity of the X chromosome non-coding RNA gene is embryonically lethal to all of the engineered male's female descendants. The terms “offspring,” “descendant,” and “descendants” are used herein in reference to post-birth organisms, not embryonic organisms.

The genetic material or “system” in the engineered male organism that knocks out the female-specific non-coding RNA gene can be introduced into a female organism of the species by impregnating the female organism with the genetic material of the engineered male. A means for the transfer of the genetic material to the female may include one or more of: a mating of an engineered male organism with a female of the organism, artificial insemination of a female of the organism with sperm obtained from the engineered organism, or an in vitro fertilization (IVF) method.

In certain methods of the invention, a female organism is in captivity when impregnated with genetic material of the engineered male organism and the offspring resulting from the pregnancy are born in captivity. In some embodiments of the invention, a captive female impregnated with genetic material of the engineered male organism is released from captivity prior to giving birth to the offspring resulting from the pregnancy. In yet other embodiments of methods of the invention an engineered male prepared using a method of the invention is released into a target population, the engineered male mates with female(s) in the population and offspring and descendants of the engineered male are produced in the population. Because of the disruption of the activity of the X chromosome non-coding RNA gene resulting from the engineered male organism's genetic material, all offspring and descendants of the engineered male are male, and all offspring fathered by descendants of the engineered male are also male.

Methods of the invention can be used to introduce into a target population of an organism, one or a plurality of an engineered male organism that comprises a system encoded on the Y chromosome that knocks out a critical sequence of an X-chromosome female-specific noncoding RNA gene and eliminating its activity. The term “introducing” as used herein in reference to a target population and a system encoded on the Y chromosome that knocks out a critical sequence of an X-chromosome female-specific noncoding RNA gene and eliminating its activity, refers to the system encoded on the Y chromosome becoming part of the genetics of organisms in a target population. The introduction of the system on the Y chromosome into the target population occurs following release of the engineered male organism or females impregnated with genetic material of the engineered male organism, and the birth of their engineered offspring. Offspring and descendants of an engineered male as described herein are capable to integrating into the target population, reproducing with members of the target population, and disseminating the system on the Y chromosome into their male offspring thereby expanding the presence of the daughterless father heritable trait in the genetics of the target population.

A population of organisms into which engineered genetic material of the invention is introduced, for example by release of engineered daughterless male organism and/or release of impregnated female organisms carrying unborn engineered offspring, may be referred to herein as a “target population.” Successive generations of organisms in a target population following introduction of genetic material that disrupts activity of an X-chromosome female-specific non-coding RNA gene, will include increasing relative numbers of male organisms versus female organisms. As the ratio shifts, the presence of fewer female organisms in the target population limits the reproductive capacity of the target population and over time reduces the number of organisms in the target population. After introducing one or more engineered male organism, or offspring thereof into a target population, the status of the gender ratio and/or numbers of organism in the population can be determined, thereby permitting assessment of the effectiveness of the introduction on the target population. Methods of the invention can be used to reduce numbers of organism in a target population but the reduction need not eliminate the population entirely. It will be understood that numbers of organism in a target population may be influenced by other events such as but not limited to: environmental conditions, immigration of organisms into the target population, resource availability, etc.

Certain embodiments of methods of the invention comprise one or more of: (1) preselecting an X-chromosomal female-specific non-coding RNA gene, that disrupting the function of which in an embryo is lethal if the embryo is female and non-lethal if the embryo is male; (2) preselecting a Y chromosomal endogenous genetic locus to engineer by introducing genetic elements/gene editing components, which may but need not be part of a translational fusion with a Y chromosome endogenous gene, wherein expression of the encoded gene editing components disrupts the function of the preselected X-chromosomal female-specific non-coding RNA gene; (3) introducing the preselected encoded genetic elements/gene editing components in a translational fusion to the preselected Y-chromosome endogenous gene in the germline of a male organism of a species to prepare an engineered daughterless male organism; (4) impregnating a female organism of the species with genetic material of the prepared engineered daughterless male organism, wherein male descendants of the impregnation are engineered daughterless males comprising the engineered Y-chromosomal endogenous gene that when expressed disrupts the preselected X-chromosomal female-specific non-coding RNA gene; and (5) releasing one or both of the impregnated female organism and the engineered daughterless male organism into a target population of the organism.

As used herein, the term “preselected” means a component of a gene editing system, including a sequence required for its appropriate expression, that is chosen for inclusion. Preselection may be based on known or expected results if a preselected component is included composition and/or in a method of the invention. For example, a particular nuclease may be preselected based on one or more characteristics, such as but not limited to: ease of expression, activity, etc. In another example, one or more guide RNAs may be preselected based on one or more characteristics of the guide RNAs. It will be understood that any component of a translation fusion composition may be independently selected. For example, in an embodiment in which there are two guide RNAs, they may be preselected based on function/activity or other characteristic, and may be preselected independent of the other. As a result, each of two or more independently preselected guide RNAs in a composition or method of the invention may be the same as the other preselected guide RNAs; of two or more independently preselected guide RNAs in a composition or method of the invention may include two or more that are the same and some that are different from others; or two or more independently preselected guide RNAs in a composition or method of the invention may each be a different guide RNA than the other preselected guide RNAs.

### Engineered Organisms and Target Populations

Certain aspects of the invention include methods for preparing engineered male organisms of a species. In some embodiments of the invention an engineered male organism is prepared that includes a system that is integrated into the Y chromosome and that removes a sequence of an X-chromosome female-specific noncoding RNA gene that is necessary for its function. The alterations generated by a system that has been introduced in the Y chromosome are lethal when present in an embryonic female of the organism, for example in any female embryo generated with genetic material from an engineered male organism. The system that has been engineered into the male organism is inherited and included in the genome of the male offspring of the engineered male organism.

A male organism to be prepared as an engineered organism for use in methods of the invention may be selected, at least in part, because the male organism has the same genetics as organisms in a target population apart from the engineered system. This feature may enhance the male organism's ability to reproduce relative to an engineered male organism with genetics less similar to the target population organism. In some embodiments of the invention an engineered male organism has not been a member of the target population and is an organism that is: naïve to the target population; transplanted into the target population from a different geographical area (even if of identical genetics); and/or transplanted in from a population of organisms that has had no contact with the target population (even if of identical genetics), etc.

Methods of the invention include preparing an engineered male organism and impregnating a female organism of the species with engineered genetic elements of the engineered male. Engineered genetic elements introduced into the Y chromosome a male organism using a method of the invention results in the removal all or a portion of a sequence of an X-chromosome female-specific non-coding RNA gene from the engineered male's X chromosome, which results in disruption of function of the non-coding RNA gene. In male embryos produced from the engineered male organism, a loss of function of the X-chromosome female-specific non-coding RNA gene may have minimal or no effect on the male embryo, but is lethal to female embryos. Thus, receiving the disrupted X chromosome female-specific non-coding RNA gene from the engineered male is lethal to female embryos but is passed through subsequent male descendants of the engineered male organism.

Certain embodiments of the invention include release into a target population of one or a plurality of engineered male organisms prepared using a method of the invention. Some embodiments of the invention include release into a target population of one or a plurality of pregnant female organisms impregnated by an engineered male organism or impregnated in a manner that delivered the engineered X chromosome in which the function of the X-chromosome female-specific non-coding RNA gene is disrupted. The term, “plurality” when used herein in reference to organisms, means more than one. In some embodiments of the invention a plurality of organisms is at least 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 1000 organisms, including all integers in the provided range. In some embodiments of the invention, a plurality is more than 1000 organisms.

As used herein, the term “target population” is a population of organisms into which genetic elements prepared using methods of the invention, for example, X-chromosomes comprising disrupted female-specific non-coding RNA gene that result in daughterless male organisms, is released. Non-limiting examples of target populations include: a wild population of the organism, an agricultural population of the organism, a human-managed population of the organism, a population of the organisms in a preserve, a population of the organisms in captivity; a zoo population of the organisms, a city population of the organisms, a rural population of the organism, etc. In some embodiments of the invention, a target population is a considered to be a pest and/or undesirable population of organisms.

It will be understood that in some embodiments of the invention a target population is the population from which the impregnated female organism was obtained prior to the impregnation, and in some embodiments of the invention the target population is a population of the same species of organism as the impregnated female, but is a different population than one from which the female organism was obtained.

Numbers, geographic distribution, and other characteristics of a target population into which one or more engineered organisms of the invention are included. Population numbers can be determined and changes assessed over time. Data on the efficacy of a release of one or a plurality of engineered males prepared using methods of the invention can be collected and assessed. Such an assessment can be used to aid in determining a number of engineered organisms of the invention to be released at one or more subsequent time points. Following release of one or more engineered daughterless male organism into a target population, methods of the invention may include determination of one or more changes in the ratio of female organisms to male organisms in the target population.

### Genetic Elements and Gene Editing Components

Certain aspects of the invention include methods of preparing one or more engineered daughterless male organisms of a species. Embodiments of methods of the invention may include delivering gene-editing components, which may also be referred to herein as “genetic elements” and/or “genetic material” into the germline of the engineered male organism of the species. Delivery of gene editing components can be performed using art-known methods. For example, using vectors, etc. and other delivery means. Some embodiments of the invention include methods for designing and/or preparing one or more compositions that can be used to introduce genetic elements into an organism. In some instances, embodiments of methods of the invention include design, construction, and/or use of one or more genetic elements, including, but not limited to: nucleic acid sequences, vector sequences, promoter sequences, and sequences encoding detectable labels, such as but not limited to fluorescent labels. Gene editing components and means of creating engineered organisms using gene-editing components are known and routinely used in the art.

Selecting one or more gene editing components to introduce into a male organism to prepare the engineered male organism, may include selecting one or more of a gene sequence, an allele, a guide RNA sequence, a nuclease, a translation fusion molecule, a delivery agent, a promoter sequence, a spacer sequence, a detectable label such as fluorescent detectable label, etc. that are appropriate to result in the engineered male organism. In some embodiments, the components are introduced to the male organism's germline. Certain aspects of the invention include use of art-known design and construction methods with which to include one or more genetic elements in an engineered male organism in a manner that they can be delivered to a female organism of the species. Some embodiments of the invention comprise introducing a Y chromosomal engineered gene from an engineered male into a female organism of a species via mating of the female with the engineered male of the invention. The term “mating” as used herein, means the action of organisms coming together to breed, for example, copulation and the transfer of genetic elements from the engineered male to the female organism of the species. In addition to mating, the presence of the genetic elements components in a germline cell can permit transfer of the Y-chromosomal engineered gene using methods such as, but not limited to: artificial insemination and in vitro fertilization—to impregnate a female of the organism with the engineered genetic elements of the invention. In some embodiments of the invention, genetic elements used in methods of the invention are delivered using an artificial means, a non-limiting example of which is use of a delivery vector.

Routine genetic engineering and gene editing methods can be used in conjunction with methods of the invention set forth herein to select and introduce one or more genetic elements in and prepare an engineered daughterless male organism, whose descendants are all engineered daughterless males. As used herein, the term “vector” used in reference to delivery of compositions of the invention refers to a polynucleotide molecule capable of transporting between different genetic environments another nucleic acid to which it has been operatively linked. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked may be referred to herein as “expression vectors.” Other useful vectors, include, but are not limited to viruses such as lentiviruses, retroviruses, adenoviruses, and phages. Vectors useful in some methods of the invention can genetically insert one or more genetic elements into a dividing or a non-dividing cell and can be used to introduce one or more compositions of the invention that comprise genetic elements into an in vivo or in vitro cell. In certain embodiments of the invention, encoded gene editing components are delivered into a germline cell of an engineered male organism and when expressed in the cell the gene editing components function to disrupt a preselected X-chromosomal female-specific non-coding RNA gene in the cell.

Vectors useful in methods of the invention may include sequences including, but not limited to one or more of a: promoter sequence; recombinase encoding sequence; enhancer sequence; additional components used in targeted recombination methods; components suitable for use in gene editing methods, non-limiting examples of which are: guide nucleic acid sequences, guide RNA sequences, sgRNA sequences, promoter sequences, DNA-binding protein encoding sequences, nuclease enzyme encoding sequences, RNA-guided nuclease enzyme encoding sequences, programmable nuclease enzyme encoding sequences, programmable base editor enzyme encoding sequences, RNA-guided base editor enzyme encoding sequences, CRISPR system components, CRISPR/Cas9 system components, 2A “ribosomal skipping” peptides, internal ribosome entry sequences; and detectable label encoding sequences, etc. Methods of the invention can be used to design and construct vectors comprising genetic elements that when delivered in a method of preparing an engineered male organism, result in the inclusion of engineered genetic materials in male descendants of the engineered male organism. Expression vectors and methods of their use are well known in the gene editing arts. Promoters that may be used in methods and vectors of the invention include, but are not limited to, cell-specific promoters or general constitutive promoters. Methods for selecting and using cell-specific promoters and general promoters are well known in the gene editing and recombinant arts. Non-limiting examples of sgRNA sequences that can be used in methods of the invention are SEQ ID NOs: 11-22, which are shown in Table 1.

TABLE 1
SpCas9 sgRNAs

| Sequence | | |
|---|---|---|
| ID Number | Name | Sequence |
| 11 | 5g1 | gctcgtttcccgtggatgtg |
| 12 | 5g2 | gtggatgtgcggttcttccg |
| 13 | 5g3 | tggatgtgcggttcttccgt |
| 14 | 5g4 | agccttatggcttatttaag |
| 15 | 5g5 | cgagatttttgacgttttga |
| 16 | 5g6 | attcttgcccatcggggcca |
| 17 | 3g1 | ttatggcttctgcgtgatac |
| 18 | 3g2 | agagcccgcgtccgccatta |
| 19 | 3g3 | cttaaactgagtgggtgttc |
| 20 | 3g4 | tactgttgctgctgatcgtt |
| 21 | 3g5 | ttgtgagttattgcactacc |
| 22 | 3g6 | aacggggcgcttggtggatg |

In some embodiments of a method of the invention, a promoter is encoded upstream of the one or more encoded guide RNAs. In some such instances, the polymerase may enhance expression of the one or more guide RNAs compared to the expression of the one or more guide RNAs in the absence of the encoded promoter. In some embodiments of the invention, the promoter is a polymerase III promoter, examples of which include, but are not limited to: a U6 promoter, a 7SK promoter, an H1 promoter, and a tRNA promoter. A promoter included in some embodiments of the invention is a synthetic promoter. A non-limiting example of which is a synthetic polymerase III promoter. A promoter included in some embodiments of the invention is a promoter native to the organism species, for example, the promoter may be native to the species of the engineered male organism. A promoter included in some embodiments of the invention is a promoter that is not native to the organism species, for example, the promoter may be a promoter that is not native to the species of the engineered male organism.

### Y-Chromosomal Expression

As described herein, a Y chromosomal locus known to permit expression of exogenous transgenes may be selected for expression of the programmable nuclease [see for example Zhao et al. 2019 Sci Rep 9, 14315 (2019)], the contents of which is incorporated herein by reference in its entirety). In one non-limiting embodiment of the invention, a constitutive enhancer/promoter may be inserted along with the nuclease gene to confer ubiquitous expression of the nuclease from the Y chromosome, which may increase the probability of disrupting target sequences. In one embodiment of the invention, the nuclease may be a Cas9 nuclease. In another embodiment of the invention, the nuclease may be a Cas12a nuclease. Guide RNAs directing nuclease activity, in one embodiment, may be expressed from polymerase III promoters encoded on one or both sides of the sequence conferring nuclease expression. In certain embodiments, the simultaneous production of nuclease and guide RNAs enables cutting and disruption of target sequences. In some embodiments, expression of the nuclease may be monitored using a marker gene, which may be expressed as a fusion to the nuclease with or without a 2A peptide. In some embodiments, all DNA sequences encoded may be combinations of those found within the body of the typical rodent.

### Y-Chromosomal Genes and Fusions

As described herein, a Y chromosomal endogenous gene may be selected for inclusion in a transcriptional or translational fusion to a gene editing system through the use of an internal ribosome entry sequence or a 2A peptide conferring ribosomal skipping. As described elsewhere herein, in some embodiments of the invention a translational fusion to an endogenous gene comprises one or more sequences encoding one, two, three, or more components of a gene editing system such as, but not limited to: a guide RNA, a nuclease, etc. In certain embodiments of the invention, a nuclease is introduced but not as part of a translational fusion to the endogenous gene. Non-limiting examples of Y-chromosomal genes that may be included in a translational fusion and used in a method of the invention are Eif2s3y, Zfy, and Ddx3y. A non-limiting example of a translational fusion to the Y-chromosomal endogenous gene that can be used in certain embodiments of methods of the invention is an N-terminal 2A-fusion to the Y-chromosomal endogenous gene.

In some embodiments of the invention, methods and compositions are provided that can be used to increase translation of an N-terminal 2A-fusion to the endogenous gene. It has been recognized that an N-terminal 2A-fusion to a gene may exhibit a reduced level of translation of the gene. Certain aspects of the invention include methods and compositions to lessen the reduction of transcription of the gene of an N-terminal 2A-fusion. Such methods of the invention may include preparing an N-terminal 2A-fusion to a gene, wherein the N-terminal 2A-fusion comprises a modified Kozak sequence. Inclusion of the modified Kozak sequence can increase the level of translation of the gene compared to the level of translation of the gene when the N-terminal 2A-fusion does not comprise the modified Kozak sequence. An N-terminal 2A-fusion that includes a modified Kozak sequence may also include a sequence that encodes a nuclease and one or more sequences encoding guide RNAs. A gene included in the N-terminal A2-fusion may be present in an organism and in some embodiments, the gene is an endogenous gene that naturally occurs in the organism. Certain embodiments of the invention include in a translational fusion an endogenous gene that is expressed in the germline of males of the organism. An example of an endogenous gene that may be used in some embodiments of the invention is Y-chromosomal gene, non-limiting examples of which are: Eif2s3y, Zfy, and Ddx3y.

Another method to increase translation of an N-terminal 2A-fusion to a gene is provided in certain embodiments of the invention in which an N-terminal 2A-fusion to a gene is prepared and the preparation comprises inserting a plurality of introns into the coding region of the gene. As a result of the method, the inserted plurality of introns increases the level of translation of the gene compared to the level of translation of the gene in the absence of the inserted plurality of introns. An N-terminal 2A-fusion comprising an inserted plurality of introns in the coding region of the gene may also include a sequence that encodes a nuclease and one or more sequences encoding guide RNAs. A gene into which is inserted the plurality of introns and is included in the N-terminal A2-fusion may be present in an organism and in some embodiments the gene is an endogenous gene that naturally occurs in the organism. Certain embodiments of the invention include in a translational fusion an endogenous gene comprising the inserted plurality of introns, wherein the gene is expressed in the germline of males of the organism. An example of an endogenous gene that may be used in some embodiments of the invention is Y-chromosomal gene, non-limiting examples of which are: Eif2s3y, Zfy, and Ddx3y. An intron inserted into the gene may be a synthetic codon, an intron that is native to the organism in which the translational fusion is present.

In some embodiments of the invention, a translational fusion to a gene can be prepared that comprises a modified Kozak sequence and comprises a plurality of introns inserted into a coding region of the gene.

### X-Chromosomal Genes

A non-limiting example of an X-chromosomal female-specific non-coding RNA gene that may be included in a method of the invention is an X-inactive specific transcript (Xist) gene. Loss of Xist function has no known effects in male organism, such as mice, nor in daughters who inherit a dysfunctional copy from their mother. However, loss of function mutations in the paternal copy of Xist are selectively lethal in embryonic daughters. In a non-limiting example, disrupted function of the Xist gene, for example loss of function of a paternal copy of Xist is lethal to female mouse embryos at embryonic day 8.5 due to failed X-inactivation in the trophoblast [Marahrens, Y., et al., (1997) Genes & Development 11 (2): 156-66; Wutz, A., et al., (2002) Nature Genetics 30 (2): 167-174; Hoki, Yuko, et al., (2009) Development 136 (1): 139-46; the content of each of which is incorporated by reference herein in its entirety).

Certain embodiments of methods of the invention include gene editing to delete all or a portion of Xist exon 1 that encodes repeated stem-loop motifs. A minimal number of encoded repeated stem-loop motifs are strictly required for X-inactivation mammals such as mice (see for example: Wutz, A., et al., (2002) Nature Genetics 30 (2): 167-174; Hoki, Yuko, et al., (2009) Development 136 (1): 139-46, the content of each of which is incorporated by reference herein in its entirety). Certain embodiments of methods of the invention that include expressing an RNA-guided nuclease and a plurality of guide RNAs that target sequences flanking or distributed throughout this region, yield a targeted deletion and loss of Xist function. An alternative method comprises use of a targeted recombinase, such as but not limited to an RNA-targeted recombinase, to excise the relevant region of the non-coding gene.

The Xist gene sequence is highly conserved across mammals, and in certain embodiments methods of the invention are used to prepare engineered daughterless male organisms of the genus Rattus, genus Mus, genus Sus, genus Felis or genus Canis. It will be understood that engineered daughterless male organisms of other genera of mammals may also be prepared using methods of the invention. In some embodiments of the invention, methods of the invention are used to prepare engineered daughterless male organisms of mammalian invasive species and pests [Loda, A. & Heard, E. (2019) PLoS Genetics 15 (9): e1008333].

A Y-chromosome-linked system of the invention can be used to disrupt Xist functionality on the X chromosome. Certain embodiments of methods of the invention include encoding a RNA-guided nuclease and multiple guide RNAs targeting sequences flanking this region, such that nuclease activity occurs in male germline cells prior to spermiogenesis. Non-limiting examples of sgRNAs that target Xist flanking regions are SEQ ID Nos: 11-22 (see Table 1). Cutting multiple sites in flanking regions and within the relevant region leads to targeted deletions when repaired by non-homologous end joining. In certain embodiments of the invention, the engineered cells include germline cells, which minimizes potential fitness costs of nuclease expression.

In some embodiments of the invention, one or more nucleases are encoded from a constitutive promoter within a genetic locus on the Y chromosome permitting ubiquitous expression, including in the male germline. In another embodiment of the invention, a germline-specific promoter may be used if activity in other tissues or developmental stages is undesirable. As a non-limiting example, the protamine promoter confers high levels of expression late in meiosis.

In some embodiments of the invention, one or more nucleases are encoded as an N-terminal 2A-peptide fusion [Trichas, G., et al., (2008) BMC Biology 6 (September): 40 and Liu, Z., et al., (2017) Scientific Reports 7 (1): 2193] to a Y chromosome-encoded protein expressed in the male germline. As a non-limiting example, the protein Eif2s3y is expressed throughout male germline development, which allows ample time for the nuclease to disrupt Xist [Mazeyrat, S., et al., (2001) Nature Genetics 29 (1): 49-53]. Methods of the invention may include a 2A peptide fusion to a Y-chromosomal gene and such embodiments of methods of the invention can result in reliable expression in the male germline [Mazeyrat, S., et al., (2001) Nature Genetics 29 (1): 49-53; Armoskus, C., et al. (2014) Brain Research 1562 (May): 23-38; Huby, Russell D. J., et al., (2014) PloS One 9 (12): e115792; and Li, Na, et al., (2016) Oncotarget 7 (10): 11321-31].

To compensate for the reduced expression of the Y-chromosomal gene resulting from inefficient translational re-initiation after the 2A peptide, in some embodiments of methods of the invention, the Kozak sequence controlling translational initiation is strengthened to the consensus sequence, and/or introns are inserted into one or more of the nuclease gene sequence to enhance transcription. Another non-limiting example of a Y-chromosomal gene expressed in the male germline that may be used in methods of the invention is Zfy2, which provides a narrow burst of expression during late meiosis. Certain embodiments of the invention preparing an engineered male organism comprises introducing one or more nucleases that are not part of a translational fusion to the Y-chromosomal endogenous gene. For example, in some embodiments of the invention, one or more nucleases introduced in a method of the invention may be encoded with its own expression signals away from existing endogenous Y chromosomal genes, although this may result in reduced activity. Embodiments of methods of the invention may include any method of expression that results in sufficient expression of the nuclease to achieve Xist deletion in the male germline. Some embodiments of the invention may include steps to reduce expression or confer tissue-specific expression in the event a practitioner desires to minimize potential fitness costs resulting from expression of the nuclease in all cells rather than only germline cells.

### Certain Applications and Embodiments

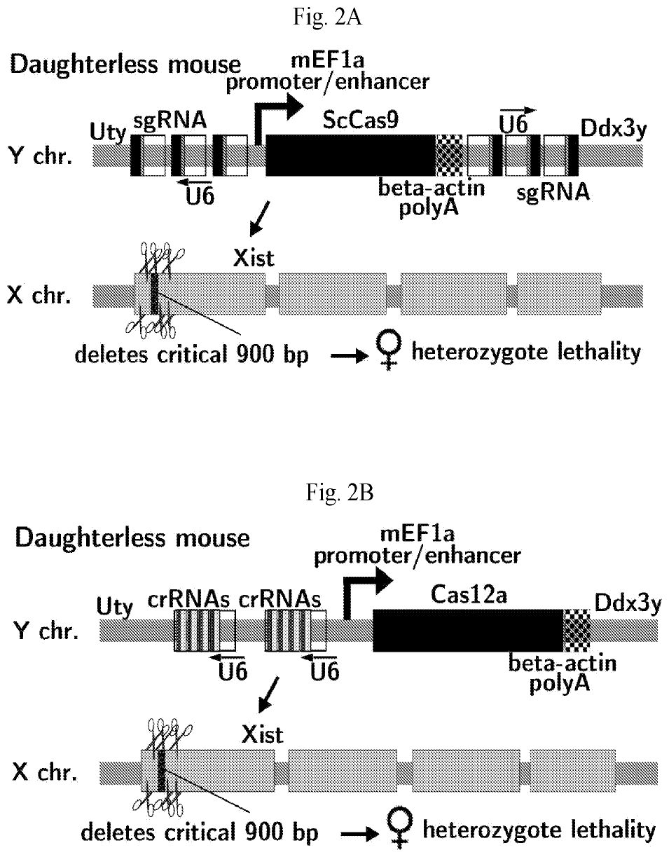

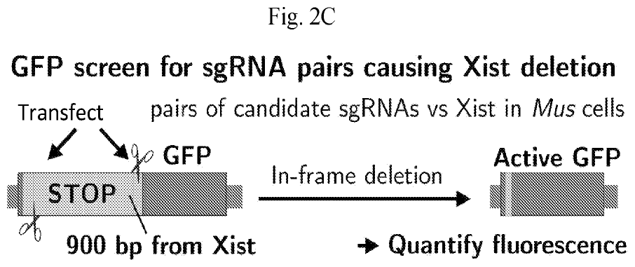

A non-limiting example of a method and compositions of the invention includes encoding an RNA-guided nuclease and multiple guide RNAs targeting sequences flanking this region, such that nuclease activity occurs in male cells prior to spermiogenesis. Cutting multiple sites in flanking regions and within the relevant region leads to targeted deletions when repaired by non-homologous end joining. Because Xist is not required at any stage of male development, the nuclease may be constitutively expressed in all cells carrying the engineered Y chromosome. For example, in one implementation, the nuclease and guide RNAs are encoded in an “open” Y-chromosomal locus known to permit the expression of inserted genes from a constitutive promoter inserted at the site, as has been demonstrated for marker genes (Zhao et al. 2019, Scientific Reports, 9: 14315). Guide RNAs are expressed from polymerase III promoters on one or both sides of the nuclease expression cassette. This architecture is detailed in FIGS. 2A and 2B.

Another non-limiting example of a method of the invention includes an implementation of compositions and methods that restrict nuclease activity to male germline cells, potentially minimizing potential fitness costs of nuclease expression. One such embodiment encodes the nuclease as an N-terminal 2A-peptide fusion [Trichas, G., et al., 2(008) BMC Biology 6 (September): 40; Liu, Z., et al., (2017) Scientific Reports 7 (1): 2193] to the Y chromosome-encoded protein Eif2s3y or Ddx3y, which are expressed in the male germline. Eif2s3y and Ddx3y are expressed throughout male germline development, allowing ample time for the nuclease to disrupt Xist (Mazeyrat et al. 2001, Nature Genetics, 29(1):49-53, Gueler et al. 2012, Human Reproduction, 27 (6): 1547-1555). A 2A peptide fusion to a germline-specific Y-chromosomal gene may also be included in some embodiments if tissue-specific expression is beneficial because it provides reliable expression in the male germline (Mazeyrat et al. 2001, Nature Genetics, 29(1):49-53; Armoskus et al. 2014, Brain research, 1562:23-38; Huby, Russell D. J., et al., PloS one, 9(12): e115792; Li et al. 2016, Oncotarget, 7(10):11321-11331). To compensate for the reduced expression of the Y-chromosomal gene resulting from inefficient translational re-initiation after the 2A peptide, the Kozak sequence controlling translational initiation is strengthened to the consensus sequence, and introns are inserted into the nuclease gene to enhance transcription. If Eif2s3y is chosen, any nonsense mutation in the sequence encoding the nuclease will be selected against due to the accompanying loss of function of Eif2s3y, which results in male sterility. In a related embodiment, the 2A peptide may be replaced with a stop codon followed by an internal ribosome entry sequence and the start codon of the subsequent gene, creating a transcriptional rather than a translational fusion.

It will be understood that paternal deposition of nuclease in sperm is not required for the success of such embodiments, nor is it harmful should this occur because Xist disruption has no effect in males. This approach does not involve sperm-killing, meaning that it is not be affected by polyandry [Manser, A. et al., (2019) Proceedings. Biological Sciences/The Royal Society 286 (1909): 20190852]. All sperm from daughterless males are equally fertile and competitive, but embryos arising from sperm that carry a female-determining X chromosome are eliminated at embryonic day 8.5, before the development of the nervous system and the onset of pain perception [Marahrens, Y., et al., (1997) Genes & Development 11 (2): 156-66].

### Release and Population Suppression

In some aspects of the invention, one or a plurality of engineered daughterless male organisms are prepared as described herein and released into a target population of the organisms. It is also envisioned that a female organism of the species may be in captivity and impregnated with engineered genetic material of the engineered daughterless male organism. Such an impregnated female organism may remain in captivity through gestation and the resulting offspring later may be released into a target population, or the impregnated female organism may be released into a target population and give birth after the release. All female embryos resulting from the impregnation with the engineered genetic material of the engineered daughterless male will die prior to birth. All descendants that result from the impregnation of the female organism with the engineered genetic material of the engineered daughterless male will be male. By reproducing in the target organism population, descendants of the engineered daughterless male organism will over time, increase the number of male organisms relative to the number of female organisms in the target population. In some embodiments of the invention, the increasing ratio of male to female organisms in the target population over time, results in fewer and fewer births in the population and a reduction in the overall number of organisms in the target population.

A target population may be a population that is in the wild or may be a captive population of the organism. In some embodiments of the invention, a population of organisms is a population not in captivity. In some instances, a population of organisms is a controlled population, for example a captive, lab-maintained population of organisms. In other instances, a population of organisms is a wild population or is a domesticated population of organisms. A target population may be a population of mammals that are considered pests and a method of the invention can be used to reduce the birth of female organism in the population and/or to reduce the total number of organism in the population.

Multiple releases of engineered daughterless male organisms and/or impregnated female organism carrying engineered embryos as described herein, into a target population are contemplated in certain aspects of the invention. Some embodiments of the invention comprise 1, 2, 3, 4, 5, 6, 7, 8, 9, 10, or more releases of one or a plurality of impregnated captive female organisms prepared using a method of the invention, into a population of organisms of the species. In some embodiments of the invention a plurality of impregnated captive female organisms of a species is released into a population of organisms of the species at 1, 2, 3, 4, 5, 6, 7, 8, or more geographic locations. In some embodiments of the invention, there are 1, 2, 3, 4, 5, 6, 7, 8, or more releases of one or a plurality of impregnated captive female organisms as described herein, into a population of organisms of the species and the number of impregnated captive female organisms and/or the number of engineered daughterless male organisms in each release may be the same or may include different numbers of the released organisms. For example, a first release may include 10 impregnated captive female organisms and a subsequent release may include 50 impregnated captive female organisms. Alternatively, a first release may include 100 engineered daughterless male organism and a subsequent release may include 50 engineered daughterless male organism. In certain embodiments of the invention factors such as: (1) the number of engineered male organisms and/or impregnated female organisms carrying unborn engineered offspring that are released into a population, (2) the geographic release location(s), (3) the timing of one or more releases of engineered male organisms and/or impregnated female organisms carrying unborn engineered offspring into a population, and/or other release characteristics are determined based on factors including but not limited to: geographic area of the population, topography of the environment that includes the population, population size, geographic range of the population, behavior of organisms of the species, and density of the population of organisms.

### Cisgenic Engineering

Another aspect of the invention relates to methods of preparing an engineered organism that is a cisgenic organism. A cisgenic organism is prepared by introducing genetic material that is one or more of: (1) derived from an organism of the same species as the organism to be engineered; (2) derived from a prokaryotic cell that is in close ecological association with the species of the organism to be engineered; and (3) derived from a eukaryotic cell that in close ecological association with the species of the organism to be engineered. Because of the origin of the genetic material, the introduction of the derived DNA fragments into the organism, results in a cisgenic organism. As used herein the terms “derived” and “derive” may be used interchangeably with the words “obtained” and “obtain”.

As used herein, the term “cisgenic” means that all DNA used to prepare an engineered organism can be found within the body of the organism that is to be engineered. This idea is supported in some cultures by what may be termed the ecological species concept: that every multicellular organism comprises both eukaryotic and prokaryotic cells, as neither set would exist without the other. Therefore, DNA can be moved from the commensal microbes of a mammal into the genomes of its eukaryotic cells (or vice versa) without being moved into a new species. Belief systems that view species according to ecological relationships rather than DNA phylogeny [Hudson, Maui, et al., (2019) Frontiers in Bioengineering and Biotechnology 7 (April): 70] may also view the commensal microbiota as one with the eukaryotic cells of the organism: since neither host cells nor commensal microbiota would function in the absence of the other, and they are always found together in the wild, they may be considered essential aspects of the same organism.

In some embodiments of the invention, an engineered daughterless male organism is prepared using only DNA found within the body of the organism to be affected by the daughterless system, inclusive of the commensal microbiota required for the organism's proper function. In a non-limiting example, a daughterless male that is cisgenic according to the ecological species concept is generated using a CRISPR nuclease from a mammalian-ubiquitous commensal microbe, such as a Cas12a gene from the Lachnospiraceae family [Zetsche, B., et al., (2015) Cell 163 (3): 759-71] or a Cas9 gene from Streptococcus canis [Chatterjee, P. et al., (2018) Sci Adv. October 24; 4(10): eaau0766. doi: 10.1126/sciadv aau0766], optionally with a native genomic sequence that functions as a 2A peptide or internal ribosome entry sequence and a species-specific polymerase III promoter for guide RNA expression, such as the U6 promoter. Any introns inserted within the nuclease gene must also be native to the species in question rather than synthetic [Tikhonov, M. V., et al., (2017) Molecular Biology 51 (4): 592-95]. These are strong, well-defined and well-understood introns taken from other genes that can tolerate the insertion of a U6 promoter and guide RNAs without creating unwanted splice sites. In some embodiments of the invention, introns are inserted within AGGT or AGGA sequences within the nuclease gene. Additional sequences within a nuclease gene are known in the art to be suitable for insertion of an intron and may be used in certain embodiments of the invention.

In some embodiments of the invention, an organism to be prepared as a cisgenic-engineered organism is a multicellular eukaryotic organism. DNA that may be introduced to the organism to prepare it as an engineered organism, may include derived DNA fragments obtained from a cell of an organism of the species of interest. The term “donor” organism may be used herein to describe an organism from which DNA fragments are obtained. Other DNA fragments that may be used to prepare a cisgenic engineered organism using a method of the invention are DNA fragments obtained from a cell that is present within the multicellular eukaryotic organism's commensal microbiota, which is also described herein as DNA fragments derived from a prokaryotic cell that is in close ecological association with the species of the organism to be engineered and/or DNA fragments derived from a eukaryotic cell that is in close ecological association with the species of the organism to be engineered.

Derived DNA fragments may be introduced to the organism to be prepared as a cisgenic engineered organism and/or two, three, four, or more of the derived DNA fragments can be spliced together and introduced to the organism as a spliced derived DNA molecule. In embodiments of methods of the invention, an introduced DNA fragment is at least 15, 16, 17, 18, 19, 20, 21, 22, 23, 24, 25, 26, 27, 28, 29, 30, or more base pairs in length. In certain embodiments of methods comprising introducing to an organism one or more spliced derived DNA molecules, the spliced derived DNA molecules may be the same or differing lengths and may be at least 30 or more base-pairs in length.

The invention in part, includes cisgenic-engineered cells. In some embodiments, a cisgenic-engineered cell is a eukaryotic cell of a species. A cisgenic engineered cell of the invention is a cell into which DNA fragments derived from one or more of: (1) an organism of the species of the cell, (2) a prokaryotic cell in close ecological association with an organism of the species of the cell, and (3) a eukaryotic cell in close ecological association with an organism of the species of the cell. In a cisgenic-engineered cell of the invention, all of the introduced DNA fragments are derived DNA fragments from only sources (1), (2), and/or (3). Methods of the invention that restrict introduced DNA fragments to (1), (2), and/or (3) can be used to prepare cisgenic-engineered cells.

In some embodiments, a cisgenic-engineered cell is a cell prepared from a cell of a multicellular eukaryotic organism. In certain embodiments of the invention, one, some, or all of the introduced DNA fragments present in a cisgenic-engineered cell are DNA fragments from a cell of a donor organism of the species of the cell. In some embodiments of the invention, one, some, or all of the introduced DNA fragments present in a cisgenic-engineered cell are DNA fragments from a cell present within the multicellular eukaryotic organism's commensal microbiota.

Derived DNA fragments are included in a cisgenic engineered cell of the invention and in some embodiments of the invention two, three, four, or more derived DNA fragments are spliced together prior to introducing the DNA into the cell. In some embodiments of cisgenic-engineered cells of the invention, an introduced DNA fragment is at least 15, 16, 17, 18, 19, 20, 21, 22, 23, 24, 25, 26, 27, 28, 29, 30, or more base pairs in length. In certain embodiments of cisgenic engineered cells of the invention, one or more spliced derived DNA molecules are introduced into the cell and the spliced derived DNA molecules may be the same or differing lengths and may be at least 30 or more base pairs in length.

### Organisms and Cells

Non-limiting examples of cells and/or stages of cells to which genetic elements, which may include gene editing components, may be delivered or included are embryonic cells, germline cells, gametes, reproductive cells, cells that can give rise to a gamete, zygotes, pre-meiotic cells, post-meiotic cells, fully differentiated cells, and mature cells. Non-limiting examples of cells into which one or more selected genetic elements may be introduced in certain embodiments of the invention, are one or more of an isolated cell, a cell in a cell line, a cell in cell culture, a cell in tissue culture, a cell in an organ culture, and a cell that is within an organism. In certain embodiments of the invention, a cell is a zygote, a gamete, a cell that is able to give rise to a gamete, a germline cell, etc.

Methods of the invention may include delivery of genetic elements, non-limiting examples of which are gene editing system components, into and implemented in various types of cells and/or organisms. As used herein the term “engineered “organism may be used to denote an organism that is prepared according to an embodiment of a method of the invention. In some aspects of the invention, a cell or organism is a vertebrate cell or organism. In certain aspects of the invention, a cell or organism is a eukaryotic or prokaryotic cell or organism. Non-limiting examples of organisms to which a method of the invention of the invention may be implemented are sexually reproducing organisms including but not limited to insects, fish, reptiles, amphibians, mammals, rodents, and birds. In some embodiments of the invention an organism is a mammal, including but not limited to cats, dogs, pigs, pigeons, starlings, fish (e.g., carp, trout, etc.), ferrets, weasels, stoats, possums, mongooses, mice, squirrels, rats, chipmunks, moles, voles, etc. In certain embodiments of the invention, an organism is not a human organism.

In some aspects of the invention, an organism of a species is selected and engineered male organism of that species are prepared because of an interest in suppressing the population number of one or more target populations of the organisms. For example, though not intended to be limiting, an urban rat population may be a target population of interest to which methods of the invention are applied to suppress and reduce the number of rats in the population.

It will be understood that methods of the invention can be used alone or used in any combination of: before, simultaneously with, and after use of one or more alternative methods to assess, monitor, reduce, suppress, and/or modulate a target population.

### Variants

Components and molecules that may be used in methods of the invention may include sequences described herein, art-known sequences and may include functional variants of such sequences. A variant nucleic acid sequence may encode a variant polypeptide that includes one or more of a deletion, point mutation, truncation, amino acid substitution, and addition of an amino acid or non-amino acid moieties, as compared to its parent polypeptide. Modifications of a polypeptide of the invention (a non-limiting example of which is an expressed genetic element) may be made by modification of the nucleic acid sequence that encodes the polypeptide. The terms “protein” and “polypeptide” are used interchangeably herein as are the terms “polynucleotide” and “nucleic acid sequence.” A nucleic acid sequence may comprise genetic element including, but not limited to RNA, DNA, mRNA, cDNA, etc. As used herein with respect to polypeptides, proteins, or fragments thereof, and polynucleotides that encode such polypeptides the term “exogenous” means the one that has been introduced into a cell, cell line, organism, or organism strain and is not naturally present in the wild-type background of the cell or organism strain. As used herein with respect to polypeptides, proteins, or fragments thereof, and polynucleotides that encode such polypeptides and functional nucleic acids, the term “endogenous” refers to a sequence that is naturally present in the genetic background of a cell, cell line, organism, or organism strain. As a non-limiting example, an endogenous Y chromosome in a mouse cell is a Y chromosome that naturally is present in the genetic background of mouse cell. In some embodiments, the term may refer to nucleic acid sequences that are typically present within the body of a multicellular eukaryotic organism, but are found within the microbial rather than the eukaryotic cells of that organism's body.

As used herein the term “variant” in reference to a polynucleotide or polypeptide sequence refers to a change of 1, 2, 3, 4, 5, 6, 7, 8, 9, 10, or more nucleic acids or amino acids, respectively, in the sequence as compared to the corresponding parent sequence. For example, though not intended to be limiting, a variant gene allele sequence may be identical to that of its parent gene allele sequence except that it includes 1, 2, 3, 4, 5, 6, 7, 8, 9, 10, or more nucleic acid substitutions, deletions, insertions, or combinations thereof, and thus is a variant of the parent gene allele. Certain methods of the invention for designing and constructing compositions for use in methods of the invention include methods to prepare functional variants of genetic element components of compositions such as encoding gene sequences, recombinase sequences, or other sequences used in methods of the invention.

Methods of the invention provide means to test for activity and function of variant sequences and to determine whether a variant is a functional variant and is suitable for inclusion in a method of the invention. Suitability can, in some aspects of methods of the invention, be based on one or more characteristics such as: expression; a resulting phenotypic trait, stability of the sequence change in offspring and descendants of a host organism, survival of an engineered male organism and/or its male descendants, etc. Functional polynucleotides that may be used in embodiments of methods of the invention may be nucleic acid sequences that have at least 85%, 86%, 87%, 88%, 89%, 90%, 91%, 92%, 93%, 94%, 95%, 96%, 97%, 98%, or 99% identity to their parent nucleic acid sequence.

Art-known methods can be used to assess relative sequence identity between two amino acid or nucleic acid sequences. For example, two sequences may be aligned for optimal comparison purposes, and the amino acid residues or nucleic acids at corresponding positions can be compared. When a position in one sequence is occupied by the same amino acid residue, or nucleic acid as the corresponding position in the other sequence, then the molecules have identity/similarity at that position. The percent identity or percent similarity between the two sequences is a function of the number of identical positions shared by the sequences (i.e., % identity or % similarity=number of identical positions/total number of positions×100). Such an alignment can be performed using any one of a number of well-known computer algorithms designed and used in the art for such a purpose. It will be understood that a variant polypeptide or polynucleotide sequence may be shorter or longer than their parent polypeptide and polynucleotide sequence, respectively. The term “identity” as used herein in reference to comparisons between sequences may also be referred to as “homology”.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1: Developing Daughterless Male Systems in Mus musculus for Local Population Suppression

Mice are invasive pests in cities, in agriculture, and in conservation that are typically controlled with rodenticides. These inhumane poisons, which frequently result in the unwanted poisoning of children and other animals (Mason G, Littin K E (2003) Animal welfare, 12(1):1-37; Brink N W van den, et al. (2018) Anticoagulant Rodenticides and Wildlife, 5:1-9; Murray M (2017) Ecotoxicology, 26(8):1041-1050), could potentially be replaced by genetic population suppression (Esvelt K M et al. (2014) eLife,: e0340; Campbell K J et al. (2015) Biol. conservation, 185:47-58). This example describes a novel implementation of the daughterless population suppression method in mice designed to be generally applicable across all mammals.

Methods are designed to be broadly useful for suppression of pests and invasive species due to the durable suppression effect following a release and the lack of a requirement for any form of gene drive. No rearrangement or mutation of a daughterless system can result in an inheritance advantage for the male-determining chromosome, necessarily precluding unwanted invasion of distant populations.

### Engineering Male-Linked Daughterless Mice

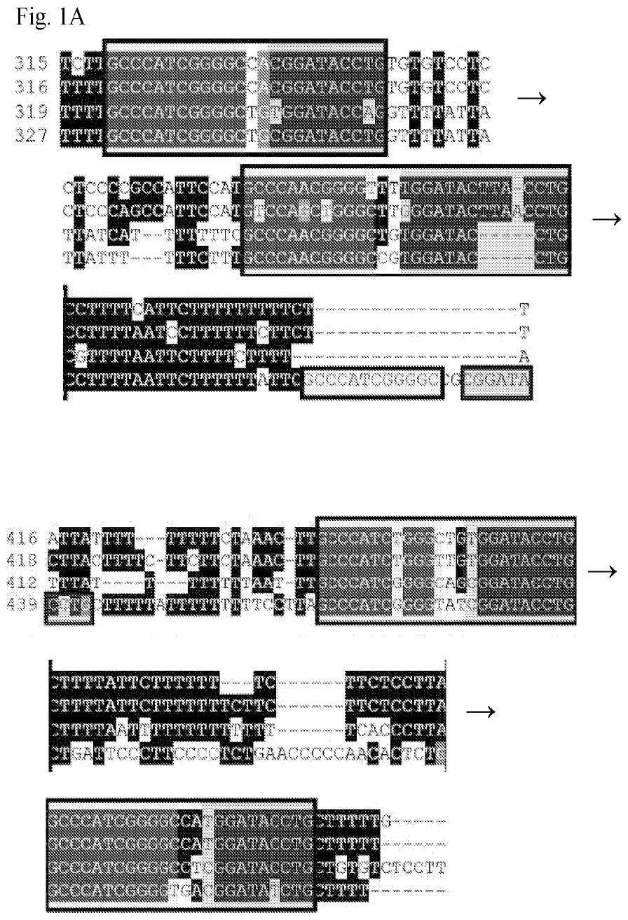

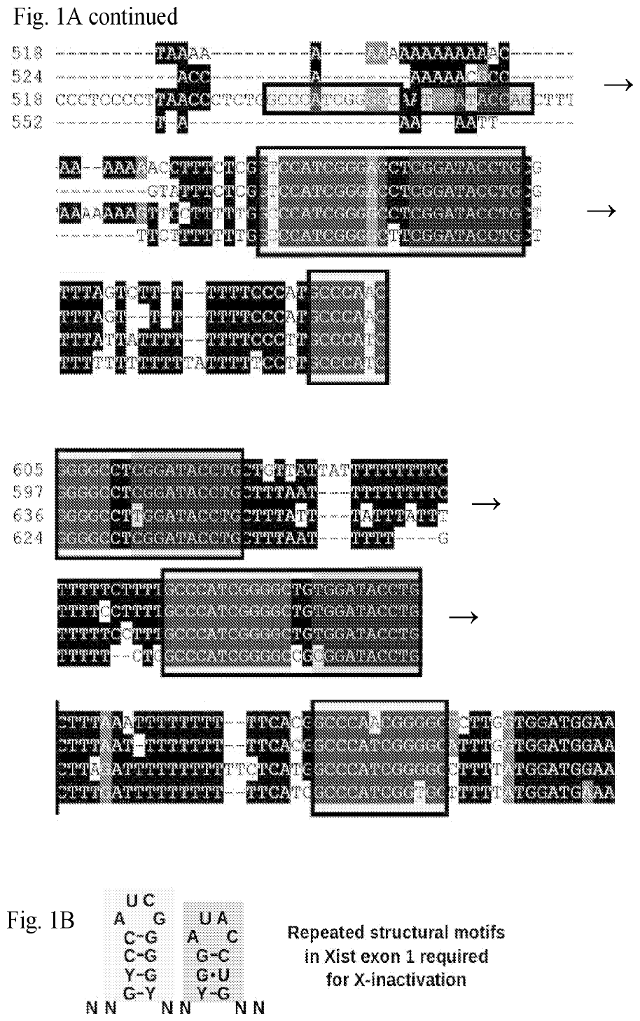

This approach involves encoding an RNA-guided nuclease and guide RNA system on the Y chromosome to knock out a critical 900 base-pair region of the female-specific noncoding RNA Xist (FIGS. 1-2). Loss of Xist function has no known effects in male mice, nor in daughters who inherit a dysfunctional copy from their mother. However, loss of function mutations in the paternal copy of Xist are selectively lethal in daughters at embryonic day 8.5 due to failed X-inactivation in the trophoblast [Marahrens, Y., et al., (1997) Genes & Development 11 (2): 156-66]; Wutz, A., et al., (2002) Nature Genetics 30 (2): 167-174; Hoki et al. 2009, Development, 136(1):139-146).

Procedures are performed to delete a small segment of Xist exon 1 that encodes critical repeated stem-loop motifs, a minimal number of which are strictly required for X-inactivation in mice (FIG. 1A-B) [Wutz, A., et al., (2002) Nature Genetics 30 (2): 167-174; Hoki et al. 2009, Development, 136(1):139-146). An RNA-guided nuclease and multiple guide RNAs targeting sequences flanking or distributed throughout this region are expressed, and yield a targeted deletion and loss of Xist function. Because Xist is highly conserved across mammals (FIG. 1A-B), this deletion method is similarly effective across rodents [Nesterova, T. B., et al., (2001) Genome Research 11 (5): 833-49] and other mammalian invasive species and pests [Loda, A. & Heard, E. (2019) PLoS Genetics 15 (9): e1008333]. There are many possible molecular implementations of a Y-linked system capable of disrupting Xist functionality on the X chromosome.

One procedure/approach includes encoding an RNA-guided nuclease and multiple guide RNAs targeting sequences flanking this region, such that nuclease activity occurs in male cells prior to spermiogenesis. Cutting multiple sites in flanking regions and within the relevant region leads to targeted deletions when repaired by non-homologous end joining.

Because Xist is not required at any stage of male development, the nuclease may be constitutively expressed in all cells carrying the engineered Y chromosome. For example, in one implementation, the nuclease and guide RNAs are encoded in an “open” Y-chromosomal locus known to permit the expression of inserted genes from a constitutive promoter inserted at the site, as has been demonstrated for marker genes (Zhao et al. 2019, Scientific Reports, 9: 14315). Guide RNAs are expressed from polymerase III promoters on one or both sides of the nuclease expression cassette. This architecture is detailed in FIGS. 2A and 2B.

Another procedure/approach restricts nuclease activity to male germline cells, potentially minimizing potential fitness costs of nuclease expression. One such embodiment encodes the nuclease as an N-terminal 2A-peptide fusion [Trichas, G., et al., 2(008) BMC Biology 6 (September): 40; Liu, Z., et al., (2017) Scientific Reports 7 (1): 2193] to the Y chromosome-encoded protein Eif2s3y or Ddx3y, which are expressed in the male germline. Eif2s3y and Ddx3y are expressed throughout male germline development, allowing ample time for the nuclease to disrupt Xist (Mazeyrat et al. 2001, Nature Genetics, 29(1):49-53, Gueler et al. 2012, Human Reproduction, 27 (6): 1547-1555). A 2A peptide fusion to a germline-specific Y-chromosomal gene is desirable if tissue-specific expression is beneficial because it provides reliable expression in the male germline [Mazeyrat et al. 2001, Nature Genetics, 29(1):49-53; Armoskus et al. (2014), Brain research, 1562:23-38; Huby, Russell D. J., et al., (2014), PloS one, 9(12): e115792; Li et al. 2016, Oncotarget, 7(10):11321-11331]. To compensate for the reduced expression of the Y-chromosomal gene resulting from inefficient translational re-initiation after the 2A peptide, the Kozak sequence controlling translational initiation is strengthened to the consensus sequence, and introns are inserted into the nuclease gene to enhance transcription. In procedures in which Eif2s3y is used, any nonsense mutation in the sequence encoding the nuclease are selected against due to the accompanying loss of function of Eif2s3y, which results in male sterility.

In another, related embodiment, the 2A peptide is replaced with a stop codon followed by an internal ribosome entry sequence and the start codon of the subsequent gene, creating a transcriptional rather than a translational fusion. In certain procedures, other Y-chromosomal genes expressed in the male germline are used in place of Eif2s3y or Ddx3y; for example, Zfy2 provides a narrow burst of expression during late meiosis.

To produce functional guide RNAs within the same cassette that encodes the nuclease in the translational fusion, polymerase III promoters expressing guide RNAs targeting Xist are embedded within introns that are inserted into the coding region of the nuclease (FIG. 2A). This approach is analogous to natural arrangements in which tRNAs are expressed from within the introns of functional protein-coding genes. While the guide RNAs could be encoded elsewhere on the Y chromosome, this arrangement permits a daughterless male to be generated with a single insertion step. Paternal deposition of nuclease in sperm is not required for the success of any of these implementations, nor is it harmful should this occur because Xist disruption has no effect in males. This approach does not involve sperm-killing, meaning that it is not be affected by polyandry [Manser, A. et al., (2019) Proceedings. Biological Sciences/The Royal Society 286 (1909): 20190852]. All sperm from daughterless males are equally fertile and competitive, but embryos arising from sperm that carry a female-determining X chromosome are eliminated at embryonic day 8.5, before the development of the nervous system and the onset of pain perception [Marahrens, Y., et al., (1997) Genes & Development 11 (2): 156-66].

### Example 2. Production of Cisgenic Daughterless Animals According to the Ecological Species Concept

Many people are uneasy with the notion of moving DNA between organisms, but are more comfortable with the idea that all DNA used can be found within the body of the organism in question. This idea is supported in some cultures by what may be termed the ecological species concept: that every multicellular organism comprises both eukaryotic and prokaryotic cells, as neither set would exist without the other. According to this view, DNA can be moved from the commensal microbes of a mammalian organism into the genomes of its eukaryotic cells (or vice versa) without being moved into a new species. Belief systems that view species according to ecological relationships rather than DNA phylogeny [Hudson, Maui, et al., (2019) Frontiers in Bioengineering and Biotechnology 7 (April): 70] may also view the commensal microbiota as one with the eukaryotic cells of the organism. At least in part because: (1) neither host cells nor commensal microbiota would function in the absence of the other, and (2) they are always found together in the wild, they may be considered essential aspects of the same organism.

The daughterless system described in Example 1, as detailed in FIG. 2A, is constructed using only DNA found within the body of the organism to be affected by the daughterless system, inclusive of the commensal microbiota required for the organism's proper function. A daughterless male that is cisgenic according to the ecological species concept is generated using a CRISPR nuclease from a mammalian-ubiquitous commensal microbe, such as ScCas9 from Streptococcus canis (Whatmore et al. 2001, 1 Clin. Microbiol. 39(11): 4196-4199). A species-specific polymerase III promoter for guide RNA expression, such as the native mouse U6 promoter for a daughterless mouse, is used. In designs including a 2A peptide or IRES is incorporated (which is not the case for FIGS. 2A and 2B), these are derived from native genomic sequences that functions as a 2A peptide or IRES, respectively. Any introns inserted within the nuclease gene are also native to the species in question rather than synthetic [Tikhonov, M. V., et al., (2017) Molecular Biology 51 (4): 592-95]. These are strong, well-defined, and well-understood introns taken from other genes that can tolerate the insertion of a U6 promoter and guide RNAs without creating unwanted splice sites. They are inserted within AGGT or AGGA sequences, or in another suitable sequence within the nuclease gene.

Native genomic sequences with 2A function present in the genome of a given species are identified through standard bioinformatics searches for DNA sequences encoding conserved 2A peptide residues, such as LLXXGDVENPGP (SEQ ID NO: 7), LLXXGDVSNPGP (SEQ ID NO: 8), LLXXXGDVENPGP (SEQ ID NO: 9), and LLXXGDVSNPGP (SEQ ID NO: 10). The genomic sequence is translated in each of the six possible bidirectional sequence frames and searched for matches to the above peptides (SEQ ID NOs: 7-10) using protein BLAST. If necessary, two native genomic DNA sequences are combined to generate a functional 2A peptide. Alternatively, the nuclease is encoded as a direct translational fusion to an endogenous protein that tolerates an N- or a C-terminal fusion. If an IRES is used, it may be a species-specific example of a cellular IRES, such as the one present in vascular endothelial growth factor and type 1 collagen-inducible protein VCIP (Licursi et al. 2011, Gene Therapy 18(6):631-6).

### Example 3. An Operational Strategy for Developing a Male-Linked Daughterless Mammal

The following describes an embodiment of a method for preparing a male-linked daughterless mammal. Non-limiting illustrations of certain embodiments are shown in FIG. 2A-B.
1. A complete genome sequence of the organism is obtained.
2. The region of Xist exon 1 containing the critical stem-loop repeats is identified.
3. Computational analysis is performed on target sites for an RNA-guided nuclease in the region.
4. Nuclease activity on the target sites and off-targets is tested in cells.
Well-performing guide RNAs are chosen based on their on-target and off-target activities.
6. A strong endogenous enhancer/promoter, such as the version of EF1a from the species in question, is identified and placed upstream of the nuclease, with a 3′ polyA tail, as from the beta-actin gene from the species in question, placed downstream.
7. Nuclease activity is verified in tissue culture using the construct to be inserted into the animal's genome.
8. Engineered organisms are generated by encoding the construct into a locus permitting expression on the Y chromosome, as between the Uty/Kdm6a and the Ddx3y genes in mice and rats.

### Example 4. Building a Daughterless Strain that is Broadly Cisgenic by the Ecological Species Concept

The following describes an embodiment of a method of preparing a cisgenic daughterless organism strain.
1. Native polymerase III promoters for guide RNA expression are identified, such as the mouse U6 promoter for daughterless Mus musculus.
2. Candidate CRISPR nucleases from the commensal microbiota of the organism are identified, such as the Cas9 protein from Streptococcus canis for many mammals.
3. The activity of the CRISPR nucleases is tested in mammalian cells using a strong enhancer/promoter and a polyA tail from genes of the species in question, thereby identifying a highly active nuclease and suitable conditions for its expression.
4. Well-performing guide RNAs for the identified CRISPR nuclease that can cut and together can excise the targeted region of Xist are chosen based on their on-target and off-target activities.
5. The identified commensal nuclease and guide RNAs, all produced using native expression signals, are encoded on the Y chromosome.

### Example 5. Screen for sgRNA Pairs Causing Xist Deletion

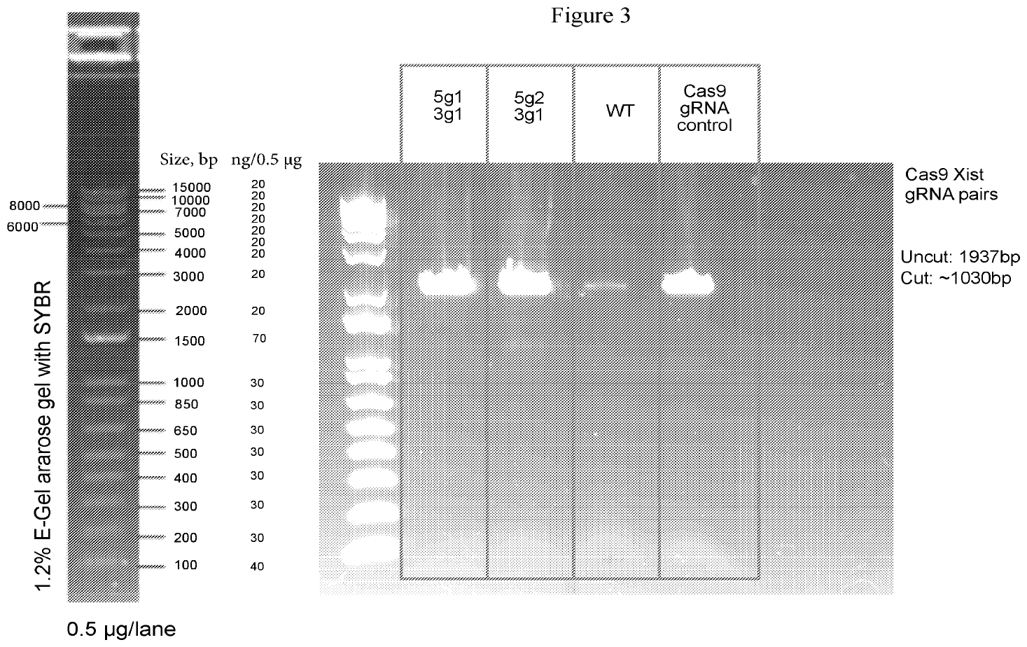

Candidate guide RNAs for Xist deletion are selected using computational analysis and then screened for activity. Target sites for an RNA-guided nuclease in the Xist region of interest are identified via computational analysis. On- and off-target nuclease activity with the candidate sgRNAs is tested, which in some instances is done in cells, and quantified (see FIG. 3).

As described elsewhere herein, the invention includes certain methods of preparing an engineered daughterless male organism of the invention comprising introducing gene editing components, such as a nuclease and/or one or more guide RNAs, etc. into a male organism. In some instances, methods include incorporating the gene editing system into the germline of the engineered male organism. An encoded nuclease and encoded guide RNAs of the gene editing system are selected, at least in part, on their ability to delete a region of an X-chromosomal female-specific non-coding RNA gene—and the resulting disruption of the X-chromosomal female-specific non-coding RNA gene function.

Studies are performed to assess guide RNA efficacy in deleting Xist gene regions. In the studies constructs encoding a CRISPR nuclease and a pair of guide RNAs are transfected into cells. The cells may be: (1) sorted by fluorescence-assisted cell sorting or (2) selected for an antibiotic resistance marker on the transfected construct (e.g. blasticidin) to isolate transfected cells. Genomic DNA is isolated from the cells by standard art-known methods and the Xist gene region amplified by PCR using primers that bind outside the guide RNA target sites flanking the region of the Xist gene that includes stem-loop repeats necessary for Xist gene function. Guide RNA pairs that result in a smaller size band are indicative of efficient deletion and identify effective pairs of guide RNAs.

Experiments performed as described above assessed the ability of various guide RNA pairs to disrupt function of the Xist gene. FIG. 3 provides results of one such study, which determined the efficacy of two guide RNA pairs, identified as 5g2/3g1 and 5g1/3g1. The results obtained for the two guide RNA pairs, as shown in FIG. 3, indicated that guide 5g2 paired with guide 3g1 was more active than guide 5g1 paired with guide 3g1. This result is evidenced by the presence of the smaller band on the gel, which resulted from deletion of the intervening DNA in the population of cells.

### Example 6. Screen for Xist-Targeting SpCas9 sgRNA Efficiency in 3T3 Cells

Methods of the invention are compatible with multiple CRISPR systems, including S. pyogenes Cas9 (SpCas9), L. bacterium ND2006 Cas12a (LbCas12a; also known as Cpf1), and S. canis Cas9 (ScCas9).

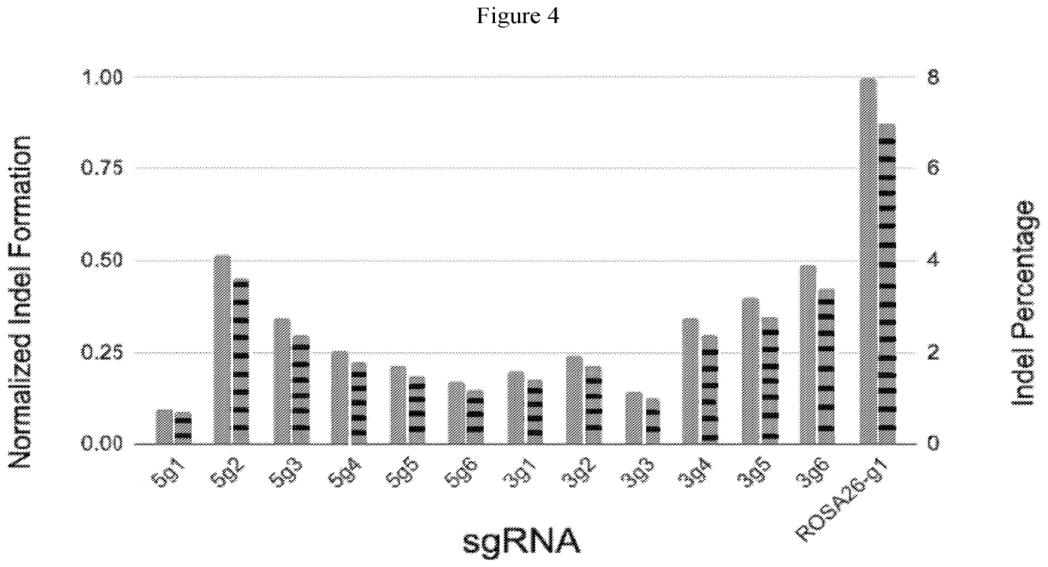

Candidate single guide RNAs (sgRNAs) for use with SpCas9 for Xist deletion were selected using computational analysis and then screened for activity. Target sites for an RNA-guided nuclease in the Xist region of interest were identified via computational analysis. Targeting efficiency of candidate sgRNAs in cells was tested and quantified (see FIG. 4). Experiments were performed as described elsewhere herein in Examples 1-5, modified as needed for use with sgRNAs, to assess the ability of SpCas9 sgRNAs to disrupt function of the Xist gene. In this study, constructs encoding an SpCas9 nuclease and an sgRNA were transfected into 3T3 cells. The cells were cultured in vitro according to standard methods.

To quantify the relative activities of sgRNAs targeting the Xist locus, a plasmid encoding the SPCas9 nuclease driven by a constitutive EF1a enhancer/promoter and a single sgRNA expressed from a human U6 promoter was transfected into NIH 3T3 cells along with another plasmid encoding tdTomato as a transfection marker. Transfections were done in duplicate and pooled for flow cytometry to isolate transfected cells for analysis. For experimental guide RNAs, region A of the mouse Xist locus featuring numerous functionally important hairpin repeats was amplified by PCR and subjected to a TIDE assay and the evaluation pipeline as originally described by Brinkmann et al 2014 Nucleic Acids Research, 2014, Vol. 42, No. 22 e 168) using the protocol detailed in Brinkman E. K., & van Steensel B. (2019) In: CRISPR Gene Editing. Methods in Molecular Biology, Vol 1961, pp 29-44. Luo Y. (eds) Humana Press, New York, NY. Indel events were identified and the indel percentage for each sgRNA was normalized against the indel percentage obtained with the control ROSA26-g1 sgRNA targeted to the ROSA26 locus. The control guide RNA targeted the Rosa26 locus.

FIG. 4 provides results of a study that determined the efficacy of 12 SpCas9 sgRNAs listed in Table 1 as SEQ ID Nos. 11-22. Results obtained for the twelve SpCas9 sgRNAs, as shown in FIG. 4, indicated that guides 5g2, 5g3, 3g4, 3g5, and 3g6 showed relatively higher editing efficiency. These results demonstrated the SpCas9/sgRNA gene editing system effectively targeted and disrupted the Xist exon 1 region.

### Example 7. Certain Embodiments of Constructs

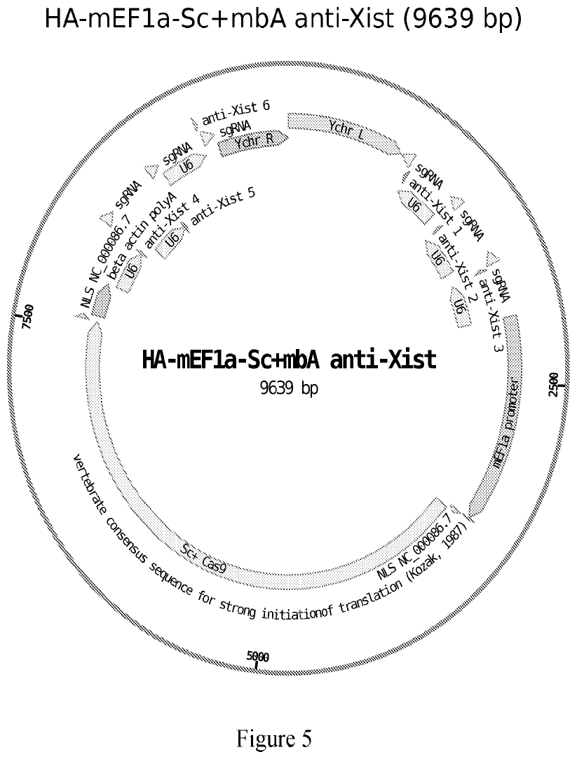

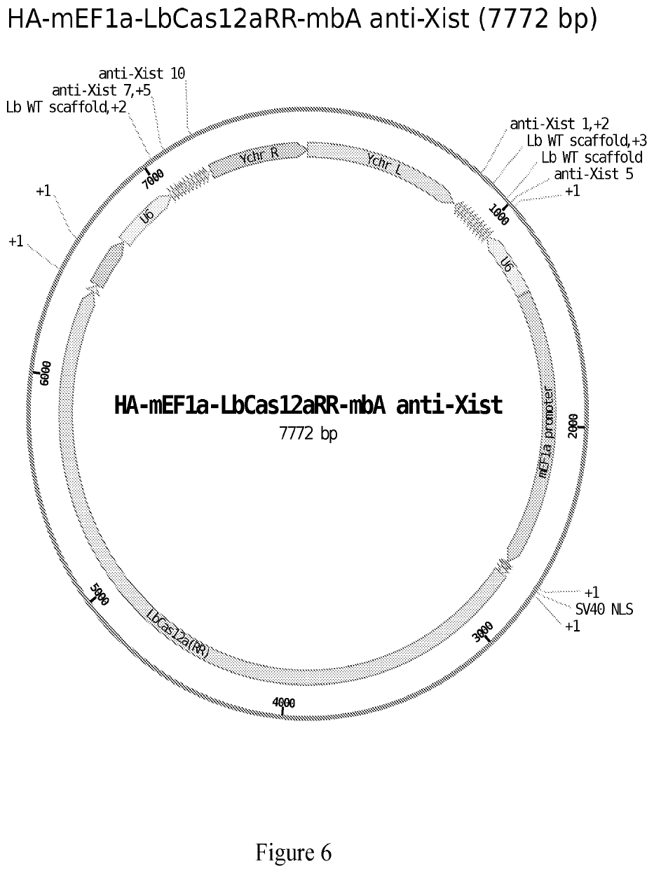

Constructs were designed, and non-limiting examples of constructs include:
(1) A 9639 bp compositional DNA construct, which is referred to herein as: HA-mEF1a-Sc+mbA anti-Xist. This construct uses a Cas9 protein from Streptococcus canis (a native rodent commensal, making it eco-cisgenic). The prepared construct is linear, but illustrated in FIG. 5 in a circular format, which permits the level of detail that is shown. FIG. 7 provides the sequence of the HA-mEF1a-Sc+mbA anti-Xist construct shown in FIG. 5.
(2) A 7772 bp compositional DNA construct, which is referred to herein referred to herein as: HA-mEF1a-LbCas12aRR-mbA anti-Xist. This construct uses a Cas12a protein. The prepared construct is linear, but illustrated in FIG. 6 in a circular format, which permits the level of detail that is shown. FIG. 8 provides the sequence of the HA-mEF1a-LbCas12aRR-mbA anti-Xist construct shown in FIG. 6.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed:

1. A composition comprising a sequence encoding a DNA nuclease programmed to target a preselected X-chromosomal gene.

2. The composition of claim 1, wherein the DNA nuclease is a Cas9 nuclease or is a Cas12a nuclease.

3. (canceled)

4. The composition of claim 1, wherein the DNA nuclease is constitutively expressed.

5. The composition of claim 1, wherein if expressed in a cell, the DNA nuclease is capable of disrupting an activity of a preselected X-chromosomal gene.

6. The composition of claim 5, wherein the preselected X-chromosomal female-specific non-coding RNA gene is an Xist gene.

7. The composition of claim 1, further comprising one or more promoter-encoding sequences capable of directing activity of the DNA nuclease to the preselected X-chromosomal female-specific non-coding RNA gene.

8. The composition of claim 7, wherein the promoter-encoding sequence is located at one or both of upstream or downstream of the sequence encoding the DNA nuclease, and optionally the encoded promoter is a constitutive promoter.

9-11. (canceled)

12. The composition of claim 1, further comprising one or more sequences each encoding a preselected guide RNA, wherein when expressed in a cell the preselected guide RNAs are capable of editing DNA of a preselected X chromosomal female-specific non-coding RNA gene.

13. (canceled)

14. The composition of claim 1, further comprising a preselected endogenous gene, wherein the preselected endogenous gene is expressed in the germline of males of the organism.

15. (canceled)

16. The composition of claim 14, wherein the preselected endogenous gene is a Y chromosomal gene.

17-20. (canceled)

21. A vector comprising the composition of claim 1.

22. A cell comprising the vector of claim 21.

23-29. (canceled)

30. A method of altering organisms of a species, comprising
(a) engineering a male organism of a species, wherein an activity of an X chromosome non-coding RNA gene is disrupted in female descendants of the engineered male organism; and
(b) producing one or more descendant organisms from the engineered male organism and a female organism of the species,
wherein the disruption of the activity of the X chromosome non-coding RNA gene in the female descendants is embryonically lethal to the female descendants.

31. The method of claim 30, wherein a means of producing the descendant organisms comprises impregnating a female organism of the species with genetic material of the engineered male.

32-33. (canceled)

34. The method of claim 30, wherein a means for the disrupting of an activity of an X chromosome non-coding RNA gene in female descendants of the engineered male organism comprises encoding on the engineered male organism's Y chromosome a nuclease capable of disrupting the activity of the X chromosome non-coding RNA gene.

35-39. (canceled)

40. The method of claim 34, wherein the nuclease is encoded in an endogenous gene in the engineered male organism, and optionally, the endogenous gene is expressed in the germline of males of the organism.

41-69. (canceled)

70. A method of increasing translation an N-terminal 2A-fusion to a gene, comprising: (a) preparing an N-terminal 2A-fusion to a gene, wherein the N-terminal 2A-fusion to the gene comprises a modified Kozak sequence, wherein the modified Kozak sequence increases a level of translation of the gene compared to a level of translation of the gene in the absence of the modified Kozak sequence; or
(b) preparing an N-terminal 2A-fusion to a gene, wherein the preparing comprises inserting a plurality of introns into the coding region of the gene, wherein the inserted plurality of introns increases a level of translation of the gene compared to a level of translation of the gene in the absence of the inserted plurality of introns.

71. The method of 70, further comprising encoding a nuclease in the N-terminal 2A-fusion, optionally wherein the nuclease is a Cas9 or Cas12a nuclease.

72. (canceled)

73. The method of claim 70, wherein the gene is in an organism, and optionally the gene is an endogenous gene of the organism.

74. (canceled)

75. The method of claim 73, wherein the endogenous gene is expressed in the germline of males of the organism, and optionally the endogenous gene is a Y-chromosomal gene.

76-129. (canceled)
